(19) United States
(12) Patent Application Publication
(10) Pub. No.: US 2018/0318454 A1
(43) Pub. Date: Nov. 8, 2018

(54) CONJUGATED POLYCATIONIC POLYMERS, METHODS OF USING THE SAME AND METHODS OF TREATING AUTOIMMUNE DISEASES, INFECTIOUS DISEASES AND ACUTE RADIATION EXPOSURE

(71) Applicant: Duke University, Durham, NC (US)

(72) Inventors: Bruce A. Sullenger, Durham, NC (US); Hemraj Juwarker, Durham, NC (US); Angelo Moreno, Durham, NC (US); Nelson Chao, Durham, NC (US); Eda Holl, Durham, NC (US)

(73) Assignee: Duke University, Durham, NC (US)

(21) Appl. No.: 15/773,765

(22) PCT Filed: Nov. 4, 2016
(86) PCT No.: PCT/US2016/060652

§ 371 Date: May 4, 2018

Related U.S. Application Data
(60) Provisional application No. 62/250,700, filed on Nov. 4, 2015.
(60) Provisional application No. 62/301,034, filed on Feb. 29, 2016.
(60) Provisional application No. 62/385,664, filed on Sep. 9, 2016.

(51) Int. Cl. A61K 49/00; A61K 31/785; A61P 31/00; A61P 37/00; A61P 43/00; A61K 47/60

(52) U.S. Cl. CPC A61K 49/0054; A61K 49/0021; A61K 31/785; A61P 31/00; G01N 33/52; A61P 43/00; A61K 47/60; A61K 2123/00; A61P 37/00

(57) ABSTRACT

Disclosed herein are polycationic polymers and methods of using the same. The polycationic polymer comprises a dendrimer or dendron, the dendrimer or the dendron comprising a focal point, a plurality of cationic termini, and a branched cationic polymer between the focal point and the plurality of cationic termini. Also disclosed polycationic polymers further comprising a detectable label; and a crosslinker, wherein the crosslinker links the detectable label and the focal point of the dendron. The resulting conjugated polycationic polymers may be used in tracking methods of methods of treating disease. Methods of using polycationic polymers to treat autoimmune diseases, infectious diseases and acute radiation syndrome are also disclosed.

### CROSS-REFERENCE TO RELATED APPLICATIONS

This patent application claims the benefit of priority of U.S. Provisional Patent Application No. 62/385,664, filed Sep. 9, 2016; U.S. Provisional Application No. 62/301,034, filed Feb. 29, 2016, and U.S. Provisional Application No. 62/250,700, filed Nov. 4, 2015, all of which are incorporated herein by reference in their entirety.

### STATEMENT OF GOVERNMENT SUPPORT

This invention was made with Government support under Federal Grant Nos. AI067798 and AI093960 awarded by the NIH. The Federal Government has certain rights to this invention.

### FIELD OF INVENTION

The invention generally relates to polycationic polymers and methods for using the same. More specifically, the invention relates to conjugated polycationic polymers and method of using polycationic polymers for treating autoimmune diseases, radiation exposure or infectious diseases.

### BACKGROUND

Nucleic acids are tasked with storing the genetic information required for life, however in many disease states nucleic acids are found in excessively high amounts and contribute to disease enhancement. This is especially true in diseases where the pathological insult is primarily from the host and not from bacteria, viruses or other common pathogens. In pancreatic cancer, for example, circulating nucleic acids are more abundant when compared to non-cancerous individuals and these nucleic acids have been shown to directly enhance disease progression ultimately adding to patient morbidity.

There have been several attempts to eliminate circulating nucleic acids from these patients and our laboratory proposes to use nucleic acid scavengers (NAS) in the form of cationic polymers in this context. Currently, the interface between scavengers and reduction in aberrant inflammation is being explored because this approach is amenable to facile chemical modification and offers an alternative to current drugs.

There exists a need for deciphering the mechanisms behind polycationic polymers and their ability to ameliorate several diseases in order to translate this approach to the clinic and for novel compounds that will help in this goal. Further, these compounds may also prove to be potential therapies on their own.

### SUMMARY OF THE INVENTION

Disclosed herein are conjugated polycationic polymers and methods of using the same. One aspect of the invention is a conjugated polycationic polymer, the conjugated polycationic polymer comprising a dendron, the dendron comprising a focal point, a plurality of cationic termini, and a branched cationic polymer between the focal point and the plurality of cationic termini; a detectable label; and a crosslinker, wherein the crosslinker links the detectable label and the focal point of the dendron. In some embodiments, the conjugated polycationic polymer is capable of binding a nucleic acid. In some embodiments, the conjugated polycationic polymer is capable of binding a nucleic acid-protein complex.

Another aspect of the invention is a scavenging apparatus, the scavenging apparatus comprising a plurality of conjugated polycationic polymers and a substrate, wherein the plurality of conjugated polycationic polymers are immobilized on the substrate. In some embodiments, the conjugated polycationic polymer is capable of binding a nucleic acid. In some embodiments, the conjugated polycationic polymer is capable of binding a nucleic acid-protein complex.

Another aspect of the invention is a method of scavenging a nucleic acid or negatively-charged biomolecule or complex from a solution, the method comprises contacting the solution comprising a cell-free nucleic acid or negatively-charged biomolecule or complex with a scavenging apparatus.

Another aspect of the invention is a method for the reduction of negatively-charged biomolecule or complex in a bodily fluid of a subject or a patient having an abnormally high concentration of the cell-free nucleic acid in the bodily fluid, the method comprising contacting the bodily fluid with a conjugated polycationic polymer or scavenging apparatus, wherein the contacting step reduces the concentration of the cell-free nucleic acid or negatively-charged biomolecule or complex in the bodily fluid.

Another aspect of the invention is a method for the tracking of a conjugated polycationic polymer, a cell-free nucleic acid, or negatively-charged biomolecule or complex in vitro or ex vivo, the method comprising contacting the conjugated polycationic polymer with a cell in vitro or ex vivo and determining the position of the conjugated polycationic polymer relative to a cell membrane or an organelle membrane of the cell, wherein the conjugated polycationic polymer is any of the conjugated polycationic polymers described above. In some embodiments, the conjugated polycationic polymer has been contacted with a negatively charged biomolecule to obtain a negatively charged biomolecule polymer adjunct and wherein step of determining the position of the conjugated polycationic polymer also determines the position of the negatively charged biomolecule polymer adjunct.

Another aspect of the invention is a method for the tracking of a negatively-charged biomolecule in vivo, the method comprising administering the negatively-charged biomolecule-polymer adjunct to a subject and determining the position of the conjugated polycationic polymer within the subject, where the conjugated polycationic polymer is any of the conjugated polycationic polymers described above. In some embodiments, the conjugated polycationic polymer has been contacted with a negatively charged biomolecule to obtain a negatively charged biomolecule polymer adjunct and wherein step of determining the position of the conjugated polycationic polymer also determines the position of the negatively charged biomolecule polymer adjunct.

### BRIEF DESCRIPTION OF THE DRAWINGS

FIG. 1 illustrates that nucleic acids released from dead and dying cells can induce pathological inflammatory responses and inflammatory diseases.

FIG. 2 shows the structures of some of the candidate polymers (n is 1 to 500).

FIG. 3 shows the monomer structures used to generate the nucleic acid-binding polymer combinatorial library. The lettered structures (A-K and AA-CC) represent the backbone.

FIG. 4 is a set of synthetic schemes for generation of the combinatorial polycationic polymer library. Michael Addition of primary or secondary amines to acrylate/acrylamide or epoxide ring opening of glycidyl ethers by primary or secondary amines was used to generate the polymers in the library. In generation of the libraries size was not a selection criterion. Thus n is, for example, 1 to 500.

FIG. 5 illustrates a synthetic approach for preparing conjugated polycationic polymers.

FIG. 6 illustrates a spectrophotometry heat map for PAMSM-AF488.

FIG. 7 illustrates a spectrophotometry heat map for PAMSM-AF750.

FIG. 8 illustrates flow cytometry of PAMSM-AF488 after incubation with macrophage cells.

FIG. 9 illustrates a spectrograph for PAMSM-AF488.

FIG. 10A shows gross appearance of skin lesions isolated 14 days after tape stripping. NZBW F1 mice treated with PBS (middle) or PAMAM-G3 (20 mg/kg, bottom) twice a week. Naïve mice (top) were not subject to tape stripping.

FIG. 10B shows representative H+E histology of skin lesions 14 days after tape stripping. Data show representative sections from 30 mice. Bars, 500 μm.

FIG. 10C shows pathologic evaluation of skin lesions post tape-stripping in naïve, PBS or PAMAM-G3 treated mice. Data are representative of four independent experiments n=12. *p<0.05).

FIG. 10D shows pathologic evaluation of skin lesions post tape-stripping in naïve, PBS or PAMAM-G3 treated mice. Data are representative of four independent experiments n=12. *p<0.05).

FIG. 10E shows pathologic evaluation of skin lesions post tape-stripping in naïve, PBS or PAMAM-G3 treated mice. Data are representative of four independent experiments n=12. *p<0.05).

FIG. 10F shows pathologic evaluation of skin lesions post tape-stripping in naïve, PBS or PAMAM-G3 treated mice. Data are representative of four independent experiments n=12. *p<0.05).

FIG. 10G shows pathologic evaluation of skin lesions post tape-stripping in naïve, PBS or PAMAM-G3 treated mice. Data are representative of four independent experiments n=12. *p<0.05).

FIG. 11A shows immune cell infiltrate in the skin of NZBW F1 mice characterized before and 24 h after tape-stripping-induced inflammation in the absence and presence of PAMAM-G3, by flow cytometry. Cell percentages are compiled and graphed as macrophages. Data are representative of 12 mice per group processed from three independent experiments.

FIG. 11B shows immune cell infiltrate in the skin of NZBW F1 mice characterized before and 24 h after tape-stripping-induced inflammation in the absence and presence of PAMAM-G3, by flow cytometry. Cell percentages are compiled and graphed as T cells. Data are representative of 12 mice per group processed from three independent experiments.

FIG. 11C shows immune cell infiltrate in the skin of NZBW F1 mice characterized before and 24 h after tape-stripping-induced inflammation in the absence and presence of PAMAM-G3, by flow cytometry. Cell percentages are compiled and graphed as neutrophils. Data are representative of 12 mice per group processed from three independent experiments.

FIG. 12A shows polycationic polymers block TLR activation by nucleic acid agonists but not LPS in DCs isolated from NZBW F1 animals similarly to wild-type mice. Polycationic polymers block IL-6 and TNFα cytokine production during CpG but not LPS stimulation in both wild-type and lupus-prone (NZBW F1) mice. Bone marrow-derived dendritic cells (DCs) were cultured as previously described. DCs were then cultured in the presence of LPS and CpG as well as 20 m/mL of each polycationic polymer (HDMBr, CDP, and PAMAM-G3). IL-6 and TNFα cytokine production was assessed 18 h later using ELISA. Data are representative of three independent experiments. n=9 mice per group. **P<0.01.

FIG. 12B shows polycationic polymers block TLR activation by nucleic acid agonists but not LPS in DCs isolated from NZBW F1 animals similarly to wild-type mice. Polycationic polymers block IL-6 and TNFα cytokine production during CpG but not LPS stimulation in both wild-type and lupus-prone (NZBW F1) mice. Bone marrow-derived dendritic cells (DCs) were cultured as previously described. DCs were then cultured in the presence of LPS and CpG as well as 20 μg/mL of each polycationic polymer (HDMBr, CDP, and PAMAM-G3). IL-6 and TNFα cytokine production was assessed 18 h later using ELISA. Data are representative of three independent experiments. n=9 mice per group. **P<0.01.

FIG. 13A shows polycationic polymers block B-cell proliferation induced by CpG but not LPS stimulation. NZBW F1 splenic B cells were isolated and carboxyfluorescein succinimidyl ester (CFSE) labeled. They were then cultured in the presence of LPS and CpG as well as 20 μg/mL HDMBr, CDP, or PAMAM-G3. Proliferation was assessed using CFSE dilution by flow cytometry. Flow plots show cell proliferation in the presence or absence of stimulation. Graph (Right) compiles proliferation data for stimulations in the presence or absence of polycationic polymer. Data are representative of three independent experiments. n=9 mice per group. *P<0.05.

FIG. 13B shows cationic polymers block IgM Ab production post CpG but not LPS stimulation. B cells were cultured in the presence of CpG and LPS as well as 20 μg/mL HDMBr, CDP, or PAMAM polymer. Supernatants were collect 72 h poststimulation, and IgM levels were assessed via ELISA. Data are representative of three independent experiments. *P<0.05.

FIG. 14A shows representative H+E histology of paraffin-embedded renal sections from PBS and PAMAM-G3 MRLlpr treated mice as well as wild type control (untreated) mice. Data show representative sections from 18 mice per group. Bars, 50 μm.

FIG. 14B shows glomerulonephritis evaluation of kidneys post PBS/PAMAM-G3 treatment. Data are from three independent experiments. Each circle represents a mouse. n=18. *p<0.05.

FIG. 14C shows frozen renal sections from PBS and PAMAM-G3 MRLlpr treated mice as well as wild type control (untreated) mice after staining for complement factor C3c. Data show representative sections from 15 mice per group. Magnification ×20.

FIG. 14D shows fluorescence intensity of the C3c staining measured using ImageJ. Data are composites of three independent experiments. Each circle represents a mouse. n=15. ***p<0.001.

FIG. 15A shows representative crithidia luciliae kinetoplast DNA slides (anti-dsDNA Abs) from serum of 20 week old MRLlpr mice treated with PBS or PAMAM-G3 and 20 week old wild type control (untreated) mice.

FIG. 15B shows representative HEp-2 ANA staining from serum of 20 week old MRLlpr mice treated with PBS or PAMAM-G3 and 20 week old wild type control (untreated) mice.

FIG. 16 shows PAMAM-G3 treatment inhibits loss of platelets from the blood of MRLlpr mice. Peripheral blood from 20-wk-old MRLlpr mice treated with PBS or PAMAM-G3 was assessed for platelet counts. Data are representative of three different experiments. n=7. *P<0.05.

FIG. 17A shows PAMAM-G3 treatment does not suppress the immune system of NZBW F1 animals during PR8 influenza infection. NZBW F1 mice were intranasally infected with PR8 influenza and injected s.c. twice per week with PBS or PAMAM-G3 (20 mg/kg). Mice were monitored for survival.

FIG. 17B shows PAMAM-G3 treatment does not suppress the immune system of NZBW F1 animals during PR8 influenza infection. NZBW F1 mice were intranasally infected with PR8 influenza and injected s.c. twice per week with PBS or PAMAM-G3 (20 mg/kg). Mice were monitored for weight loss.

FIG. 18 shows PAMAM-G3 treatment does not affect the ability of NZBW F1 mice to mount a germinal center response after a sublethal dose of PR8 influenza treatment. Spleens from PR8 influenza-infected NZBW F1 mice were isolated 14 d posttreatment with PBS or PAMAM-G3. Percentages of GL7+B220+B cells were determined by flow cytometry. Data are representative of two independent experiments. n=4.

FIG. 19A shows PAMAM-G3 treatment protects C57BL6/J mice from lethal PR8 influenza infection. C57BL6/J mice were intranasally infected with PR8 influenza (PR8) or treated with saline (mock) and injected intraperitoneally twice per week with PBS or PAMAM-G3 (20 mg/kg). Mice were monitored for survival. n=7 per group. Graphs are representative of at least three independent experiments. **P<0.01.

FIG. 19B shows treated mice monitored for weight loss throughout the study.

FIG. 19C shows Anti-influenza neutralizing Ab titers from infected mice analyzed by microneutralization assay.

FIG. 20 shows PAMAM-G3 treatment does not affect the ability of wild-type mice to mount a germinal center response after lethal dose of PR8 influenza treatment. Spleens from PR8 influenza-infected C57BL/6 mice were isolated 14 d post treatment with PBS or PAMAM-G3. Percentages of GL7+B220+B cells were determined by flow cytometry. Data are representative of two independent experiments. n=5.

FIG. 21 shows survival probability of mice treated with PAMAM-G3 or PBS vehicle subjected to lethal irradiation.

FIG. 22 is a set of graphs and photographs demonstrating that the cationic polymers are capable of rescuing mice from lethal nucleic acid-induced inflammatory shock. FIG. 22A is a graph showing the percentage of mice surviving over time after challenge with D-GalN and CPG. FIG. 22B is a graph showing the percentage of mice surviving over time after challenge with D-GalN and Poly I:C. FIG. 22C is a set of photographs of hemotoxylin and eosin stained liver sections of mice injected with PBS, CpG 1668+D-GalN or CpG 1668+D-GalN+NAS CDP.

FIG. 23 is a set of photographs and graphs showing the interaction of the conjugated polycationic polymers with DNA in neutrophils and the protective effect of administration of conjugated polycationic polymers to scavenge DAMPS and protect exposed cells from TLR activation after radiation exposure. FIG. 23A is a set of photographs showing the interaction of a biotinylated polycationic polymer with DNA in neutrophils. The biotinylated polymer is stained red, the DNA is stained green and areas of overlap are yellow (see arrows). FIG. 23B shows a graph of TLR activation after radiation exposure of cells followed by addition of polycationic polymer attached to beads to scavenge inflammatory mediators in the cell culture media. FIGS. 23C and E are similar experiments to that shown in FIG. 23B but use patient serum collected before and after radiation. FIG. 23D is a graph showing the protein levels in the patient sera. FIG. 23F is a graph showing the DNA levels present in the sera.

### DETAILED DESCRIPTION

Nucleic acid molecules were thought to be largely immunologically inert until it was discovered that the innate immune system employs pattern-recognition receptors (PRRs) to recognize various molecular patterns associated with harmful pathogens and damaged cells and to initiate inflammatory responses. In particular, various bacterial or viral derived DNA and RNA molecules were found to activate several PRRs, including at least four different Toll-like receptors (TLR3, 7, 8 and 9) and several cytoplasmic PRRs.

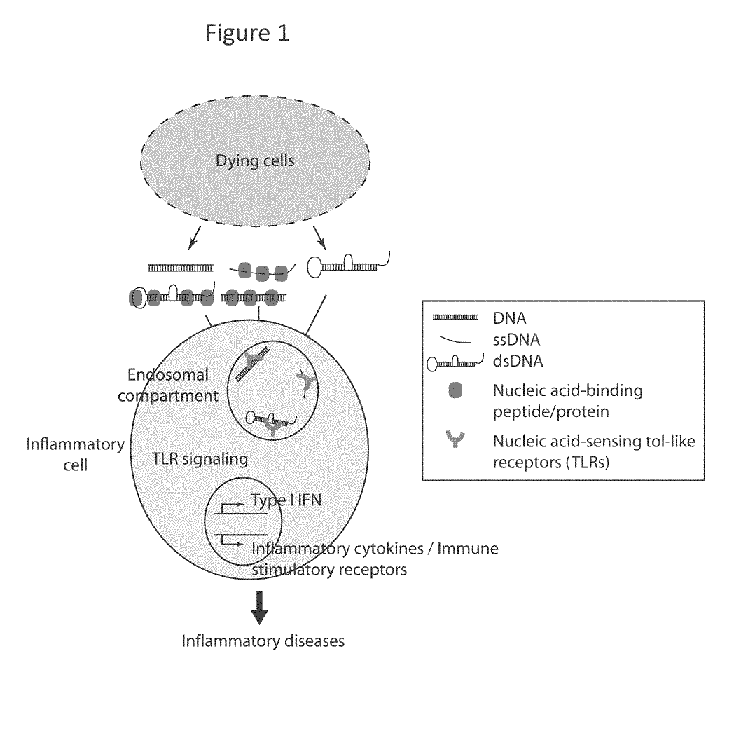

Given their potent immunostimulatory and proinflammatory effects, the discovery that the inappropriate activation of TLRs/PRRs is associated with a broad range of inflammatory disorders was not a surprise. More specifically, it now appears that nucleic acid-sensing TLRs play a critical role in numerous inflammatory disorders presumably because dead and dying cells release nucleic acids and nucleic acid-containing complexes into the extracellular space which induces pathogenic inflammatory responses (FIG. 1).

Moreover, increased levels of extracellular circulating mtDNA have even been associated with increased mortality in Intensive Care Unit patients. Thus, nucleic acid-sensing TLRs and PRRs have become attractive therapeutic targets for the treatment of acute pathological inflammation as well as devastating inflammatory disorders. Unfortunately the redundancy of the TLR and PRR families as well as their ability to sense a variety of structurally different nucleic acid ligands has made it challenging to develop effective inhibitors that can broadly ameliorate the proinflammatory effects of RNA, DNA and nucleic acid-containing complexes. Moreover, because these PRRs are important for responding to infectious agents, therapeutic strategies that compromise TLR function (or their downstream effector molecules) compromise an animal's or patient's ability to combat infection. Novel anti-inflammatory agents that do not affect innate immunity toward pathogenic infection while being able to mitigate the effects of inflammation are required.

We demonstrate that polycationic polymers can inhibit the activation of nucleic acid-sensing TLRs (TLR3, 7, 8 and 9) and the inflammatory response engendered by prototypical proinflammatory nucleic acids in vitro as well as rescue animals from nucleic acid-induced fatal inflammatory shock. Moreover we show that conjugated polycationic polymers can be used to detect and sequester nucleic acids. As a result polycationic polymers may be used to treat conditions associated with elevated levels of cell-free nucleic acids, such as autoimmune diseases, infectious diseases, or acute radiation syndrome, by neutralizing the effects of proinflammatory and procoagulant nucleic acid-based DAMPs released from damaged cells while at the same time not compromising the native immune systems ability to combat infectious diseases.

### Polycationic Polymers

Polycationic polymers, which are sometimes referred to as nucleic-acid scavenging polymers, are polymers having a plurality of cationic termini, a focal point or bridging moiety, and a branched cationic polymer between the focal point or the bridging moiety and the cationic termini. The polycationic polymers may be a dendrimer or a dendron.

Dendrimers or dendrons may be characterized by the generation number Gn. The generation number details the number of successive additions of the polymers base monomer. The generation number (Gn) may characterize the dendron's properties depending on the choice of the polymer. Properties characterizable by knowledge of the generation number and the cationic polymer include, without limitation, the number of branch points, the size of the dendron, the electronic charge, and terminal moieties. In certain embodiments, the dendron is a G2 dendron, a G3 dendron, a G4 dendron, a G5 dendron, G6 dendron, or any Gn suitable for use as a scavenger.

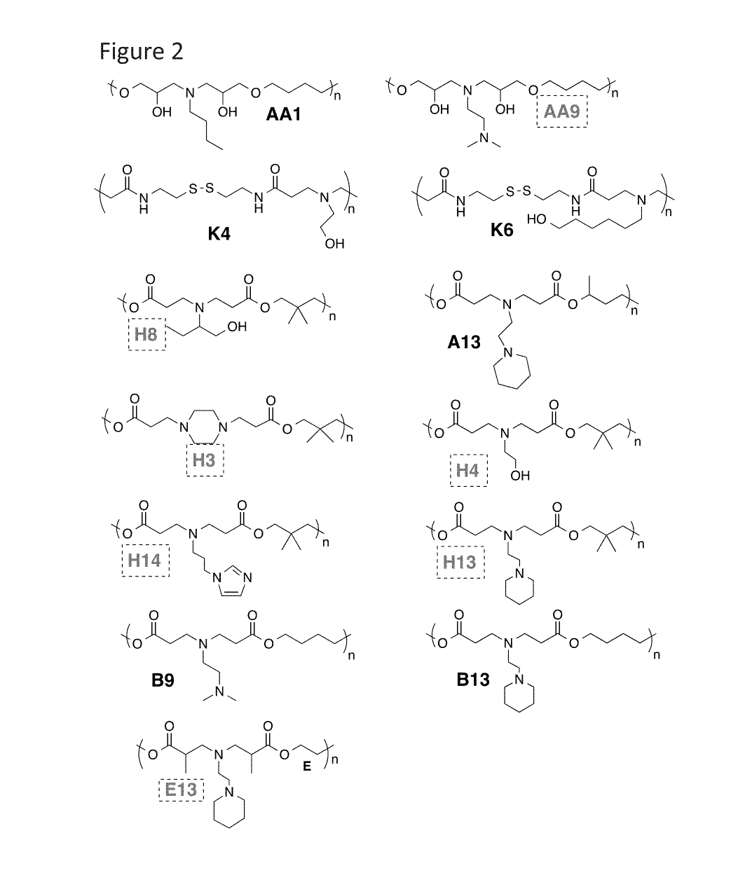

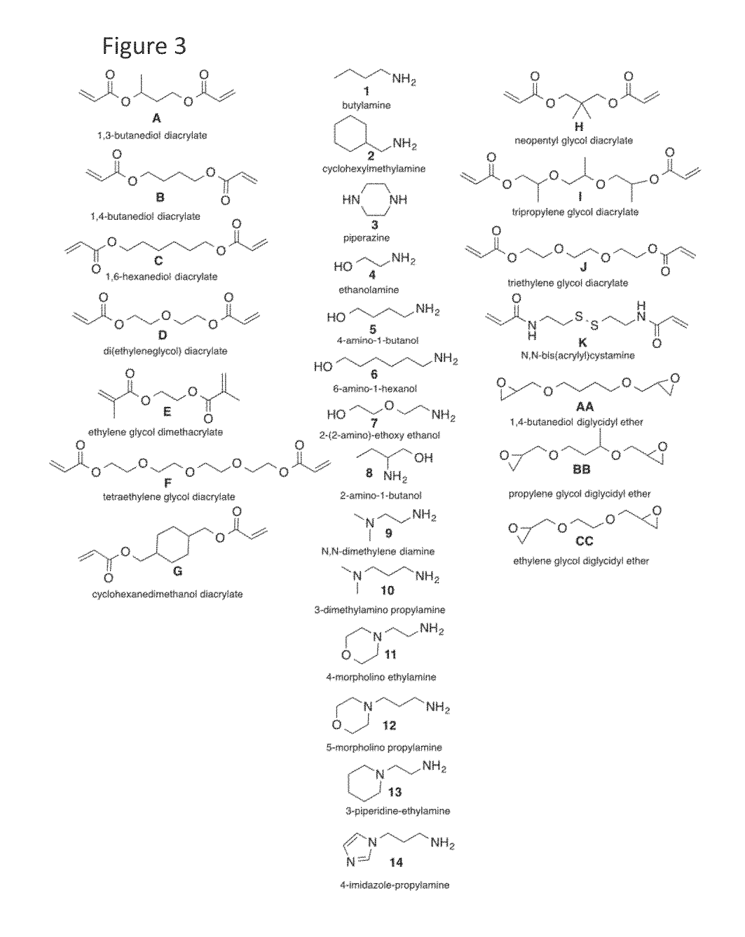

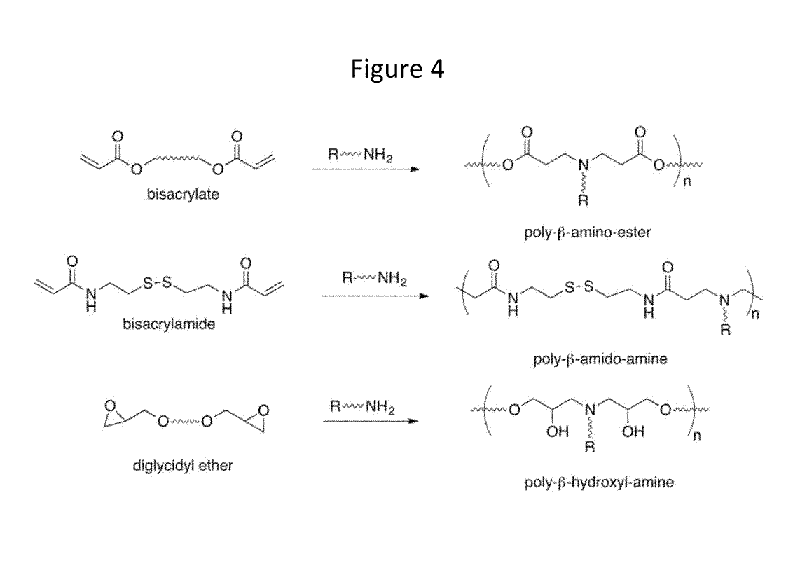

In some embodiments, the polycationic polymer is selected from the group consisting of a poly(β amino ester), disulfide containing poly(β amido amine) or poly(β hydroxyl amine). Preferred polymers include those in FIG. 2, particularly preferred are AA9, H3, H4, H8, H13 and H14 where “n” is, for example, 1 to 500, preferably, 5 to 250, more preferably, 10-200, 20-150 or 30-100. Other suitable polymers include A1, A2, A6, A9, A13, A14, B5, B6, B8, B9, B13, E13, F6, F8, F9, H2, H3, H4, H6, H7, H8, H9, H13, H14, I1, I13, K4, K6, K9, K14, AA1, AA9, and BB1. For each of the listed polymers the backbone is the structure listed as A-K or AA-CC as shown in FIG. 3 and the monomer side chain has the structure indicated as 1-14 in FIG. 3. The polymers are made from the monomers shown in FIG. 2 using the reactions shown in FIG. 4 to generate the polymers listed. From the results in the Example the most suitable backbone polymers were A, B, H, K and AA and the most suitable side chain monomer linkers were 1, 6, 8, 9, 13 and 14. Cationic polymers of the invention include biodegradable and non-biodegradable polymers and blends or copolymers thereof. Several of these are further exemplified in International publication No. WO2014/169043.

The polycationic polymer is suitably a polycationic polymer capable of binding to a nucleic acid. Preferred polycationic polymers include biocompatible polymers (that is, polymers that do not cause significant undesired physiological reactions) that can be either biodegradable or non-biodegradable polymers or blends or copolymers thereof. PAMAM G3 was used in the examples, but other polycationic polymers are anticipated to achieve similar effects. Examples of such polymers include, but are not limited to, polycationic biodegradable polyphosphoramidates, polyamines having amine groups on either the polymer backbone or the polymer side chains, nonpeptide polyamines such as poly(aminostyrene), poly(aminoacrylate), poly(N-methyl aminoacrylate), poly(N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,N-dimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride); natural or synthetic polysaccharides such as chitosan, cyclodextrin-containing polymers, degradable polycations such as poly[alpha-(4-aminobutyl)-L-glycolic acid] (PAGA); polycationic polyurethanes, polyethers, polyesters, polyamides, polybrene, etc. Particularly preferred cationic polymers include CDP, CDP-Im, PPA-DPA, PAMAM and HDMBr. (See U.S. Pat. Nos. 9,340,591, 7,270,808, 7,166,302, 7,091,192, 7,018,609, 6,884,789, 6,509,323, 5,608,015, 5,276,088, 5,855,900, U.S. Published Appln. Nos. 2012/0183564, 20060263435, 20050256071, 200550136430, 20040109888, 20040063654, 20030157030, International Patent Publication No. WO 2014/169043, Davis et al, Current Med. Chem. 11(2) 179-197 (2004), and Comprehensive Supramolecular Chemistry vol. 3, J. L. Atwood et al, eds, Pergamon Press (1996).)

The plurality of cationic termini may be any terminal moieties that allow for the binding of negatively charged molecules. The polycationic polymer may bind nucleic acids or other negatively charged molecules to the corona of a dendrimer or dendron. Under certain conditions, the plurality of cationic termini may assist to effectively bind the nucleic acid irreversibly. Under certain under condition, the plurality of cationic termini may assist to effectively bind the nucleic acid reversibly. The plurality of cationic termini may be an ammonium terminal moiety or any other cationic termini suitable for binding to nucleic acids.

Advantageously, the binding affinity of a polycationic polymer of the invention for a nucleic acid, expressed in terms of Kd, is in the pM to mM range, preferably, less than or equal to 50 nM; expressed in terms of binding constant (K), the binding affinity is advantageously equal to or greater than 10⁵ M⁻¹, preferably, 10⁵ M⁻¹ to 10⁸ M⁻¹, more preferably, equal to or greater than 10⁶M⁻¹. Thus, the binding affinity of the sequence-independent nucleic acid-binding cationic polymers can be, for example, about 1×10⁵ M⁻¹, 5×10⁵ M⁻¹, 1×10⁶ M⁻¹, 5×10⁶ M⁻¹, 1×10⁷ M⁻¹, 5×10⁷ M⁻¹; or about 10 μM, 100 pM, 1 nM, 10 nM, 100 nM, 1 μM, 10 μM, 100 μM. “K” and “Kd” can be determined by methods known in the art, including Isothermal calorimetry (ITC), Forster Resonance Energy Transfer (FRET), surface plasmon resonance or a real time binding assay such as Biacore.

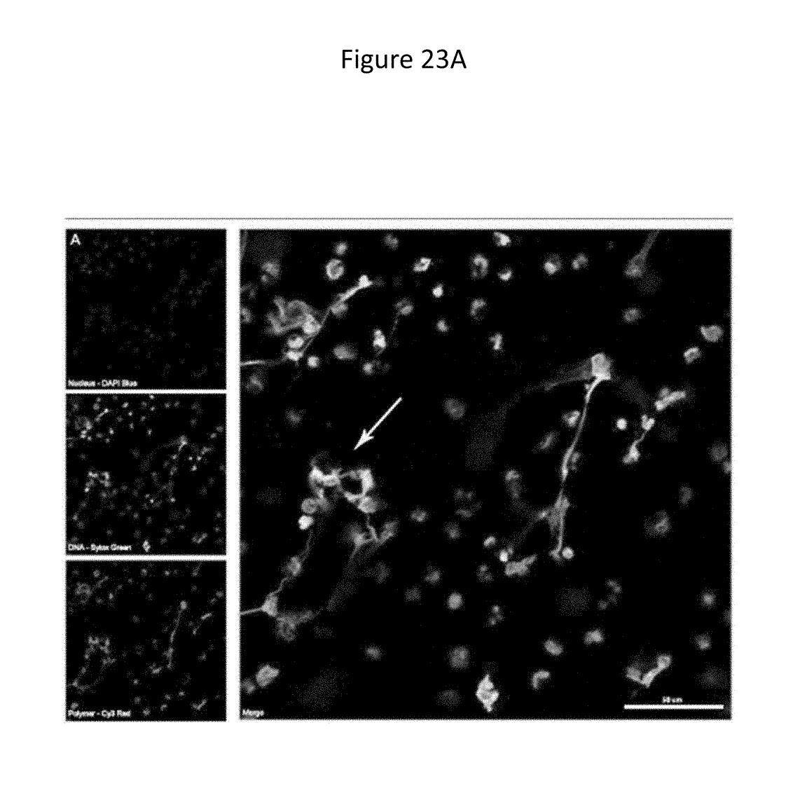

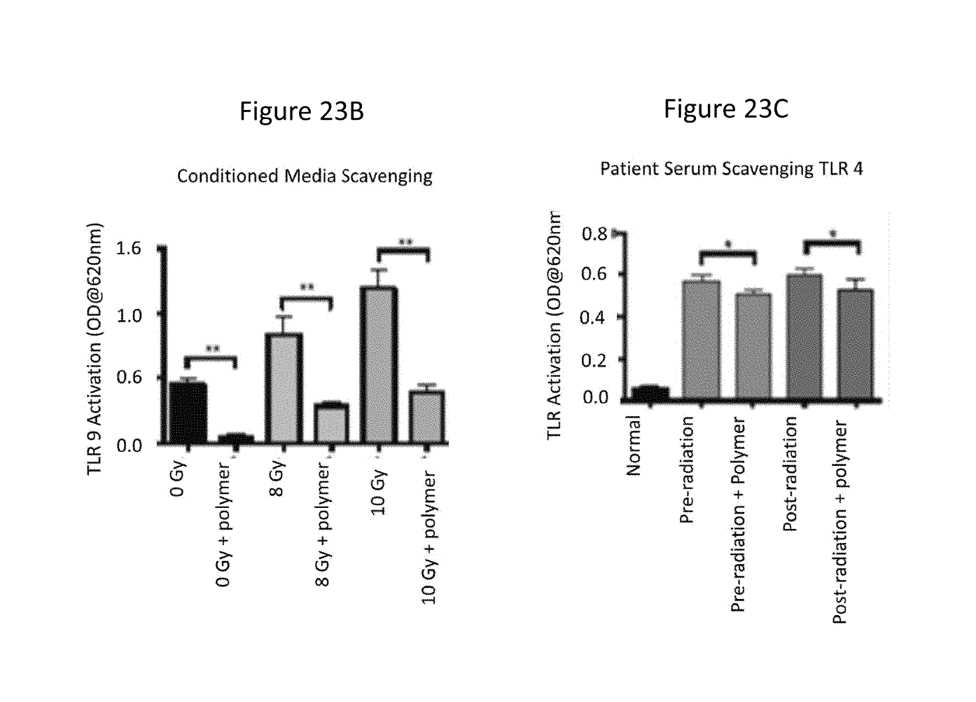

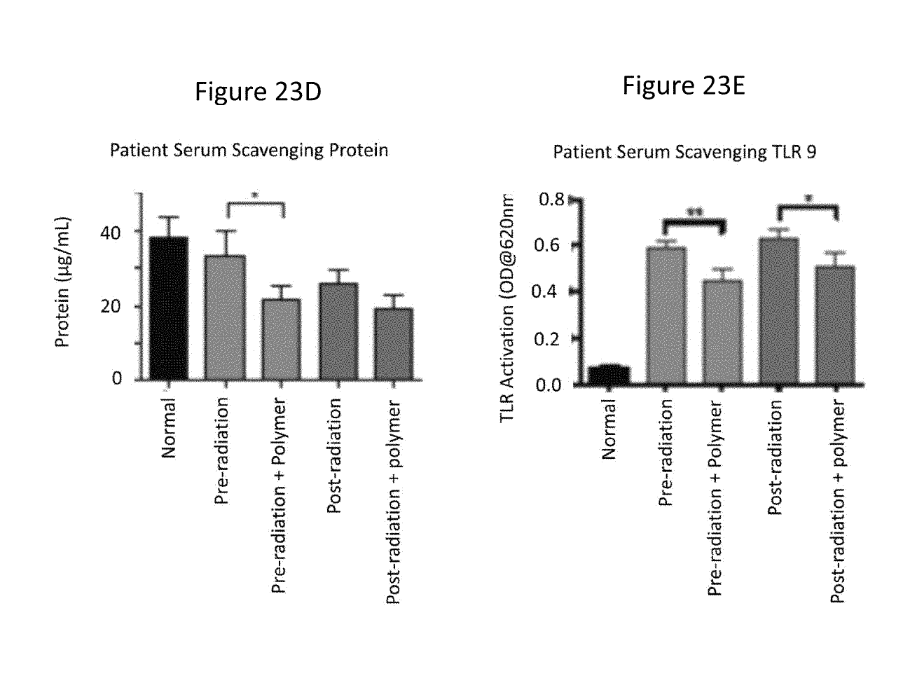

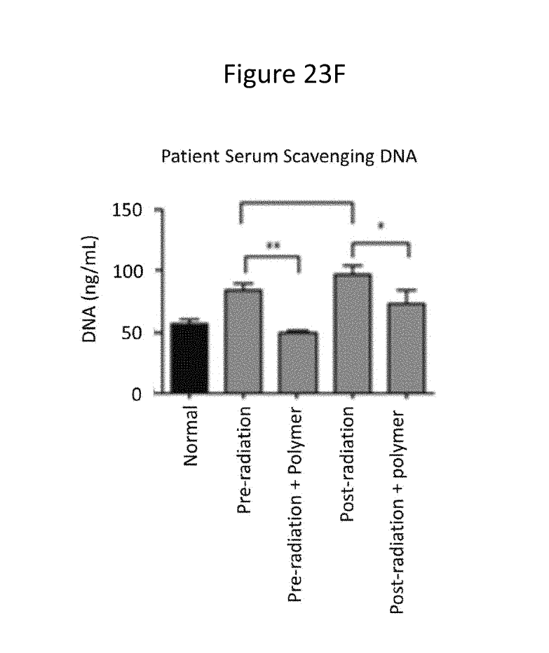

The cationic polymers bind to a wide array of different nucleic acids including ssRNA, ssDNA, dsRNA and dsDNA and of which may be presented in a complex with protein such as viral proteins, histones, HMGB1 or RIG-I. See FIG. 23. The polycationic polymer also binds DAMPs (damage associated molecular pattern) and PAMPS (pathogen-associated molecular pattern) as well as other inflammatory mediators.

Conditions such as pH, presence or absence of salts, and/or temperature may affect the electronic character of the polycationic polymer and within the scope of the invention. Depending on the conditions for using the polycationic polymer, the plurality of termini or the branched polymer between a focal point or a bridging moiety and the plurality of termini may be electrically neutral. Under some conditions, the polycationic polymer has a plurality of electrically neutral termini and a branched cationic polymer between a focal point or a bridging moiety and the plurality of electrically neutral termini. Under some conditions, the polycationic polymer has a plurality of cationic termini and a branched electrically neutral polymer between a focal point or a bridging moiety and the plurality of cationic termini.

### Conjugated Polycationic Polymers

One aspect of the invention is conjugated polycationic polymers. The conjugated polycationic polymers comprise a dendron having a focal point, a plurality of cationic termini, and a branched cationic polymer between the focal point and the plurality of cationic termini, a detectable label, and a crosslink that links the detectable label and the focal point of the dendron. The conjugated polycationic polymers have the ability to bind to negatively charged molecules, such as nucleic acids or nucleic acid-protein complexes, to sequester the negatively charged molecules and/or prepare a trackable adjunct.

In some embodiments, the crosslinker is prepared by contacting a first crosslinkable moiety with a second crosslinkable moiety. The dendron may further comprise the first crosslinkable moiety and the detectable label comprises a second crosslinkable moiety, and the first crosslinkable moiety is capable of crosslinking with the second crosslinkable moiety. The first crosslinkable moiety and/or the second crosslinkable moiety may be a sulfhydryl, carbonyl, carboxyl, amine maleimide, haloacetyl, pyridyl disulfide, thiosulfonate, vinylsulfone, hydrazide, alkoxyamine, carbodiimide, isothiocyanates, isocyanates, acyl azides, N-Hydroxysuccinimide ester, sulfonyl chloride, glyoxal, epoxide, oxirane, carbonate, aryl halide, imidoester, carbodiimide, anhydride, and fluorophenyl ester, or any other crosslinkable moiety.

The detectable label may be a binding label, a chromophore, an enzyme label, a bioluminescent label, a quencher, a radiolabel, or any other label suitable for a means of detection. Binding labels provide for a detectable signal via a binding event. In some embodiments, a binding label may be biotin, an antibody, an antigen, or any other label capable of providing a detectable signal via a binding event. Chromophores provide a detectable signal via the absorbance and emission of photons. In some embodiments, the chromophore is a fluorophore, a phosphor, a dye, a quantum dot, or any other chromophore capable of absorbing and emitting detectable photons. In certain embodiments, the chromophore is an Alexa Fluor such as Alexa Fluor 488 or Alexa Fluor 750. Enzyme labels provide a detectable signal via a reaction with a substrate. Bioluminescent labels provide a detectable signed via the emission of light from a protein. In certain embodiments, the bioluminescent label is a luciferase. Quenchers provide a detectable signal via the modulation of the photon emission from a chromophore. Radiolabels provided for a detectable signal via a radioactive decay.

### Apparatuses for Binding and Sequestering Negatively Charged Molecules

Another aspect of the invention is a polycationic apparatus for binding and sequestering negatively charged molecules. The polycationic apparatus comprises a plurality of conjugated polycationic polymers and a substrate, wherein the plurality of conjugated cationic polymers are capable of being immobilized on the substrate. In certain embodiments, the conjugated polycationic polymers are any of the conjugated polycationic polymers described above.

In an embodiment of the apparatus, the substrate comprises a binding moiety and the detectable label binds with the binding moiety to immobilize the polycationic polymer polymer on the substrate. The binding moiety may be avidin, an antibody, or any other binding protein. When avidin is used as a binding moiety, the detectable label is an avidin-binding label. In particular embodiments when avidin is used as a binding moiety, the detectable label is biotin. When an antibody is used as a binding moiety, the detectable label is an antibody-binding label. In particular embodiments when an antibody is used as a binding moiety, the detectable label may be an antigen.

The binding moiety may also be a binding moiety that binds a protein. In particular embodiments, the binding moiety may be biotin or an antigen. When biotin is used as a binding moiety, the detectable label may be a biotin-binding label. In particular embodiments when biotin is used as a binding moiety, the detectable label is avidin. When an antigen is used as a binding moiety, the detectable label may be an antigen-binding label. In particular embodiments when an antigen is used as a binding moiety, the detectable label is an antibody.

In an alternative embodiment of the apparatus, the plurality of polycationic polymers is covalently bound to the substrate. The polycationic polymers comprise a dendron, the dendron comprising a focal point, a plurality of cationic termini, and a branched cationic polymer between the focal point and the plurality of cationic termini, and a crosslinker, wherein the crosslinker links the substrate and the focal point of the dendron. The dendron may further comprises a first crosslinkable moiety, the substrate comprises a second crosslinkable moiety, the second crosslinkable moiety capable of crosslinking with the first crosslinkable moiety; and the crosslinker is prepared by contacting the first crosslinkable moiety with the second crosslinkable moiety. In certain embodiments, the first crosslinkable moiety or the second crosslinkable moiety comprises a member selected from the group consisting of sulthydryl, carbonyl, carboxyl, amine maleimide, haloacetyl, pyridyl disulfide, thiosulfonate, vinylsulfone, hydrazide, alkoxyamine, carbodiimide, isothiocyanates, isocyanates, acyl azides, N-Hydroxysuccinimide ester, sulfonyl chloride, glyoxal, epoxide, oxirane, carbonate, aryl halide, imidoester, carbodiimide, anhydride, or fluorophenyl ester.

The substrate may be any substrate suitable for binding the polycationic polymer. In certain embodiments, the substrate may be a glass, silicon, a silicon polymer, a metal, a plastic, magnetic, or an electrospun fiber. Glasses may include silica, a borosilicate, soda lime, or any other glass suitable for binding the polycationic polymer. Silicone polymers may include polydimethylsiloxane or any other silicone polymer suitable for binding the polycationic polymer. Metals may include gold, silver, platinum, or any other metal suitable for binding the polycationic polymer. Plastics may include a poly(methyl methacrylate), a poly(styrene), cyclic olefin copolymer, or any other plastic suitable for binding the polycationic polymer. Magnetic substrates may include any magnetic material suitable for binding the polycationic polymer, including, magnetic beads. The electrospun fiber may be any electrospun fiber suitable for binding the polycationic polymer, including those described in International Application Ser. No. PCT/US2015/026201 to Sullenger et al., published as WO/2015/161094 22 October 2015. Those skilled in the art will appreciate that there may be many ways to immobilize the polycationic polymer to the substrate depending on the choice of substrate.

Sequestering Negatively Charged Molecules from Solutions or Biological Samples

Another aspect of the invention is methods for scavenging negatively charged molecules, such as a nucleic acid, from a solution or a biological sample. The method comprises contacting the solution comprising a negatively charged molecule with any of the apparatuses described above. In certain embodiments, the apparatus comprises the conjugated polycationic polymers also described above. The solution may be artificially created by human intervention or a biological sample obtained from a subject or a patient. When the solution is a biological sample obtained from a subject or a patient, the solution may be blood, lymph, plasma, serum, cerebral spinal fluid, urine or any other bodily fluid. In certain embodiments, the solution or biological sample comprises cell-free nucleic acids.

In one embodiment of the invention, the conjugated polycationic polymer is bound to the substrate of the apparatus and the solution or biological sample is contacted with the bound conjugated polycationic polymer. When the solution or biological sample is contacted with the bound conjugated polycationic polymer, the cationic polymer may bind negatively charged molecules to prepare an adjunct. By forming the adjunct, the negatively charged molecules will be sequestered by the bound conjugated polycationic polymer.

In an alternative embodiment, the conjugated polycationic polymer is deposited into the solution or biological sample and the solution or sample containing the conjugated polycationic polymer is contacted with the substrate of the apparatus. By depositing the conjugated polycationic polymer into the solution or biological sample, you allow for the formation of adjuncts between the conjugated polycationic polymer and negatively charged molecules present. When the adjuncts are later contacted with the apparatus, the adjuncts may bind to the substrate through the conjugated polycationic polymers. This, in turn, sequesters the negatively charged molecules.

### Detecting the Conjugated Polycationic Polymers or Negatively-Charged Biomolecules or Complexes, Including Cell-Free Nucleic Acids

Another aspect of the invention includes methods for the tracking the conjugated polycationic polymer or a negatively charged biomolecule or complex, such as cell-free nucleic acid, in vitro or ex vivo. The method comprises contacting the polycationic polymer adjunct with a cell in vitro or ex vivo; and determining the position of the conjugated polycationic polymer relative to a cell membrane or an organelle membrane of the cell. In some embodiments, the conjugated polycationic polymer may be contacted with a negatively charged biomolecule or complex to prepare an adjunct that allows for simultaneous detection of the negatively charged biomolecule or complex via the adjunct. When practicing the method, it may be possible to determine that the polycationic polymer adjunct is bound to the cell membrane or the organelle membrane. When practicing the method, it may also be possible to determine that the polycationic polymer or polycationic polymer adjunct is determined is within the cell membrane or the organelle membrane. Such methods may be useful for determining whether or not the polycationic polymer and/or negatively charged molecule enter cells. If so, the methods may also be useful for determining the rate of uptake through a number of different analytical tools, including, but not limited to, flow cytometry and confocal microscopy. Such methods may also be useful for screening cell or tissue types for the ability to internalize polycationic polymers and/or prone to polycationic polymer toxicity. Further still, such methods may be able to determine if polycationic polymers localize with cellular organelles or other intracellular compartments.

Another aspect of the invention includes methods for the tracking the conjugated polycationic polymer or a negatively charged biomolecule or complex, such as a cell-free nucleic acid, in vivo. The method comprises administering the polycationic polymer to a subject and determining the position of the polycationic polymer within the subject In some embodiments, the conjugated polycationic polymer may be contacted with a negatively charged biomolecule or complex to prepare an adjunct that allows for simultaneous detection of the negatively charged biomolecule or complex via the adjunct. The administering step may comprise intravenous injection, intraperitoneal injection, subcutaneous injection, or any other suitable method of administration. Practicing the method may also allow for the determination of whether the polycationic polymer adjunct is within a tissue of the subject. This allows for the determination of the polycationic polymer localization. This further allows for the determination of pharmacokinetics, including, but not limited to rate of clearance and biological binding capacity. This method also opens up the ability to analyze which routes of administration results in more rapid degradation of the polycationic polymer.

Either of the in vitro, ex vivo, or in vivo tracking methods may use a detectable label as described above. In some embodiments, the detectable label is a chromophore. In certain embodiments, the detectable label is a fluorophore. In particular embodiments, the detectable label is an Alexa Fluor, for example Alexa Fluor 488 or Alexa Fluor 750. When the detectable label is a chromophore, the determining step may comprise exciting the detectable label and detecting the localized position of emitted photons. The position of the nucleic acid may be determined by any suitable method. Examples of methods and/or techniques for determining position, include, but are not limited to, fluorescence spectroscopy, fluorescence microscopy, confocal microscopy, flow cytometry, fluorescence-activated cell sorting, or immunohistochemistry.

### Methods of Using Nucleic-Acid Scavenging Polymers for Therapeutic Applications

Another aspect of the invention provides methods for the reduction of cell-free nucleic acid or other inflammatory mediator in a bodily fluid of a subject or a patient having an abnormally high concentration of the cell-free nucleic acid or other mediator of inflammation in the bodily fluid. The method comprises contacting the bodily fluid with a polycationic polymer or nuc scavenging apparatus, wherein the contacting step reduces the concentration of the cell-free nucleic acid, DAMPS, PAMPS or other inflammatory mediators in the bodily fluid. The polycationic polymer may be any of the conjugated polycationic polymers described above or any of the unconjugated polycationic polymers described above. The scavenging apparatus may be any of the scavenging apparati described above. In certain embodiments, the contacting step is performed within the subject or the patient. In other embodiments, the contacting step is performed outside the subject or the patient. The method may further comprise obtaining the bodily fluid from the patient and/or returning the bodily fluid to the subject or the patient. In some embodiments, the bodily fluid is blood, plasma, serum, cerebral spinal fluid, lymph, or any other bodily fluid having cell-free nucleic acids or other inflammatory mediators.

In certain embodiments, the subject or patient suffers from a condition associated with the abnormally high concentration of the cell-free nucleic acid or other inflammatory mediators. The condition may be a cancer, an effect associated with radiation therapy, an autoimmune disease, an infectious disease, or any other condition associated with abnormally high concentrations of cell-free nucleic acid or other inflammatory mediators in a bodily fluid. In particular embodiments, practicing the methods described herein may provide therapeutic benefit for the subject or patient suffering from the condition.

The polycationic polymer may be used to make pharmaceutical compositions. Pharmaceutical compositions comprising the polycationic polymers described above and a pharmaceutically acceptable carrier are provided. A pharmaceutically acceptable carrier is any carrier suitable for in vivo administration. Examples of pharmaceutically acceptable carriers suitable for use in the composition include, but are not limited to, water, buffered solutions, glucose solutions, or oil-based carriers. Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, dextran), proteins such as albumin or casein, protein-containing agents such as bovine serum or skimmed milk and buffers (e.g., phosphate buffer). Especially when such stabilizers are added to the compositions, the composition is suitable for freeze-drying or spray-drying. The composition may also be emulsified.

The polycationic polymer may be administered with an addition therapeutic agent. The polycationic polymer and therapeutic agent may be administered in any order, at the same time or as part of a unitary composition. The two may be administered such that one is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks or more. The polycationic polymer may be administered or used to contact a bodily fluid of the subject in conjunction with another therapy to treat the disease or condition.

An effective amount or a therapeutically effective amount as used herein means the amount of the polycationic polymer that, when administered to a subject for treating the condition is sufficient to effect a treatment (as defined above). The therapeutically effective amount will vary depending on the compositions or formulations, the disease and its severity and the age, weight, physical condition and responsiveness of the subject to be treated.

The compositions described herein may be administered by any means known to those skilled in the art, including, but not limited to, oral, topical, intranasal, intraperitoneal, parenteral, intravenous, intramuscular, subcutaneous, intrathecal, transcutaneous, nasopharyngeal, intratumoral or transmucosal absorption. Thus the compounds may be formulated as an ingestable, injectable, topical or suppository formulation. The compositions may also be delivered within a liposomal or time-release vehicle. Administration to a subject in accordance with the invention appears to exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage administered in any given case will be adjusted in accordance with the compositions being administered, the disease to be treated or inhibited, the condition of the subject, and other relevant medical factors that may modify the activity of the compound or the response of the subject, as is well known by those skilled in the art. For example, the specific dose for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compound of the invention and of a known agent such as tocopherol, such as by means of an appropriate conventional pharmacological or prophylactic protocol. The subject may be a human subject, a human suffering from cancer or a non-human animal subject. For example, the subject may be a domesticated animal such as a cow, pig, chicken, horse, goat, sheep, dog or cat.

The maximal dosage for a subject is the highest dosage that does not cause undesirable or intolerable side effects. The number of variables in regard to an individual prophylactic or treatment regimen is large, and a considerable range of doses is expected. The route of administration will also impact the dosage requirements. It is anticipated that dosages of the compositions will reduce symptoms of the condition at least 10%, 20%, 30%, 40%, 50%, 60%, 70%, 80%, 90% or 100% compared to pre-treatment symptoms or symptoms is left untreated. It is specifically contemplated that pharmaceutical preparations and compositions may palliate or alleviate symptoms of the disease without providing a cure, or, in some embodiments, may be used to cure the disease or disorder.

Suitable effective dosage amounts for administering the compositions may be determined by those of skill in the art, but typically range from about 1 microgram to about 50,000 micrograms per kilogram of body weight weekly, although they are typically about 50,000 micrograms or less per kilogram of body weight weekly. Large doses may be required for therapeutic effect and toxicity of the compositions is likely low. In some embodiments, the effective dosage amount ranges from about 10 to about 50,000 micrograms per kilogram of body weight weekly. In another embodiment, the effective dosage amount ranges from about 100 to about 40,000 micrograms per kilogram of body weight weekly. In another embodiment, the effective dosage amount ranges from about 500 to about 30,000 micrograms per kilogram of body weight weekly. The effective dosage amounts described herein refer to total amounts administered, that is, if more than one compound is administered, the effective dosage amounts correspond to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

### Methods of Using Nucleic-Acid Scavenging Polymers for Treating Autoimmune Diseases

Autoimmune disorders such as systemic lupus erythematosus (SLE) are characterized by increased production of antibodies against self-nucleic acids and their associated proteins. A number of Toll-like receptors (TLRs) that assist in recognition of these nucleic acids have been individually targeted to slow and reverse disease progression. These therapeutic strategies have shown an essential role for TLR-targeting in combating inflammatory disorders, however, their effects to date have been modest and new drugs remain to be tested.

Endosomal TLRs act as sensors of foreign RNA and DNA and elicit an innate immune response to pathogens. When tolerance is broken, these TLRs are aberrantly activated by self-nucleic acids; often associated with autoantibodies and immune complexes. This in turn leads to increased downstream activation of signaling cascades and dysregulated expression of pro-inflammatory cytokines and autoantibodies. Despite our increased understanding of TLR biology and attempts to target these particular pathways, we have been unable to develop effective ways to address the source of antigen.

Inappropriate clearance of dying cells and elevated levels of serum DNA and RNA correlates with increased autoimmune disease pathology. Studies have shown that targeting the free-circulating RNA and DNA using nucleases can be beneficial in dampening inappropriate TLR activating and improving autoimmune disease outcome.

We directly tested the hypothesis that polycationic polymers can limit pathological inflammation during the course of autoimmunity, by absorbing free-circulating DNA and RNA or nucleic acid-protein complexes. We utilized two mouse models of systemic lupus erythematosus (SLE), NZBWF1 and MRLlpr, to assess immune activation and resolution of inflammation in the presence of polycationic polymers. Both mouse strains develop spontaneous SLE, which closely mimics clinical human SLE. In addition, we further explored the hypothesis that polycationic polymers deliver a therapeutic benefit during autoimmunity without compromising the organism's ability to fight infections. To test this hypothesis we employed a PR8 influenza infection in the presence of polycationic polymers.

Here we show that polycationic polymers are capable of controlling aberrant inflammation in two separate disease models. polycationic polymers treatments resulted in improved skin inflammation and also delayed systemic lupus progression. Additionally, we demonstrated that mice treated with polycationic polymers were capable of responding to pathogenic infections such as PR8 influenza. Moreover, polycationic polymers treatment of mice during a lethal PR8 influenza infection resulted in increased survival rates. It is important to note that our studies utilized a widely used polycationic polymer: generation-3 PAMAM-G3, [NH2(CH2)2NH2]:(G=3);dendri PAMAM(NH2)32, a cationic polymer (MW6909) with a core of 1,4-diaminobutane. This molecule contains 32 surface amine groups, which allows for high affinity binding of nucleic acids, an important property that results in better polycationic polymer efficacy.

Taken together these results suggest that targeting nucleic acids with polycationic polymers could present a therapeutic strategy not only for autoimmune disorders but also for treatment of dangerous acute inflammation. We expect that the results shown for lupus will extend to other autoimmune disease wherein inflammation is associated with the pathology of the autoimmune disease, including but not limited to, psoriasis, rheumatoid arthritis, inflammatory bowel disease, multiple sclerosis, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid and associated skin diseases, cardiomyopathy, Celiac diseae, Celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, cutaneous necrotizing venulitis, discoid lupus, erythema multiforme, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic/autoimmune thrombocytopenia purpura (ITP), immunologic lung disease, immunologic renal disease, IgA neuropathy, juvenile arthritis, lichen planus, lupus erythematosus, Ménière's disease, mixed connective tissue disease, type 1 or immune-mediated diabetes mellitus, myasthenia gravis, pemphigus-related disorders (e.g., pemphigus vulgaris), pernicious anemia, polyarteritis nodosa, polychrondritis, polyglandular syndromes, polymyalgia rheumatica, polymyositis and dermatomyositis, primary agammaglobulinemia, primary biliary cirrhosis, psoriatic arthritis, Raynauld's phenomenon, Reiter's syndrome, rheumatic fever, sarcoidosis, scleroderma, Sjögren's syndrome, stiff-man syndrome, spondyloarthropathies, systemic lupus erythematosis (SLE), lupus erythematosus, systemic vasculitis, takayasu arteritis, temporal arteristis/giant cell arteritis, thrombocytopenia, thyroiditis, ulcerative colitis, uveitis, vasculitides such as dermatitis herpetiformis vasculitis, vitiligo, and Wegener's granulomatosis.

Inflammation is a complex biological process that is necessary for clearance of pathogens. However, when acute inflammation turns chronic, it can lead to inflammatory disorders that are hard to control. Dead or dying cells release RNA and DNA into circulation. If these self-nucleic acids are not properly cleared, they can trigger activation of endosomal TLRs such as TLR7, 8 and 9. This in turn results in further downstream activation of signaling pathways and production of pro-inflammatory cytokines. In fact, multiple autoimmune disorders are characterized by elevated levels of circulating pro-inflammatory cytokines and auto-antibodies. To date, numerous studies and clinical trials have focused on addressing circulating self-RNA and DNA, TLR activation, proinflammatory cytokines and circulating auto-antibodies. Many of these drugs act on a single component or cell type of the inflammatory response, have short-term effects and do not break the TLR activation cycle. Additionally, a number of these compounds are associated with increased susceptibility to infection and decreased pathogen clearance.

The goal of our study was to break the cycle of aberrant inflammation by targeting self-nucleic acids prior to their binding of endosomal TLRs. To address our goals, we utilized a novel class of compounds, scavengers, which bind circulating nucleic acids and block TLR activation. Treatment of immune cells from both CD57/B16 wild type animals and NZBWF1 lupus prone animals with nucleic acid agonists in the presence of polycationic polymers resulted in diminished pro-inflammatory cytokine production (IL6 and TNF-a) in vitro. Cell activation through non-endosomal TLRs remained intact, thus further demonstrating the specificity of our compounds for nucleic acids. Importantly, these compounds inhibited nucleic acid-driven TLR activation in cultures of DCs derived from SLE-prone animals, suggesting that these compounds can potentially be effective in an autoimmune disease setting.

Polycationic polymers administered local and systemically in lupus prone animals improved disease outcomes in these animals. Endogenous nucleic acid-driven inflammation was diminished in the presence of polycationic polymers. In CLE models we observed reduced skin inflammation which resulted in improved disease pathology. Moreover, long-term SLE studies in the presence of polycationic polymers demonstrated the potential of these compounds to reduce levels of circulating auto-antibodies as well as decreased organ damage due to uncontrolled inflammation.

Chronic and unresolved inflammation is often associated with reduced platelet counts in patients as disease severity is increased. Here we demonstrate that treatment of lupus prone mice with polycationic polymers rescues platelet depletion thus making these compounds an important therapeutic agent, capable of acting on several different components of the inflammatory cascade as well as several different cell types.

TLR targeting has been attempted using several inhibitory compounds which bind directly to TLR7 and TLR9. Although direct inhibition of endosomal TLRs can in principle result in improved autoimmune disease outcomes, there is room for improvement. Nucleic acids are capable of activating a cohort of endosomal receptors, including but not limited to TRL7 and TLR9, thus rendering these compounds partially effective in multifaceted autoimmune disorders that rely on multiple types of receptors. Moreover, TLR7 gene mutations play an important role in disease severity and any therapies that directly target these receptors could potentially fail due to decreased mutant receptor binding. Our strategy does not rely on receptor binding, therefore, addressing a number of these concerns. We used PAMAM-G3 as the polycationic polymers in these studies due to its 32 surface amines that allow for high affinity binding of nucleic acids while lower generation PAMAM dendrimers were not as effective at inhibiting nucleic acid-mediated TLR activation in our previous in vitro studies with model TLR ligands. Additionally, generation 5 PAMAM has revealed increased toxicity in our previous and current studies at a similar dosing regimen. However our studies suggest that exploration of higher generation dendrimers with biodegradable properties or other polycationic polymers are warranted as they may further improve treatment outcomes. Moreover, a drug delivery device could prove to be an important strategy to deliver therapeutic agents slowly and uniformly and thereby increase their efficacy while eliminating any toxicity.

### Methods of Using Scavenging Polymers for Treating Infection Diseases

Lastly, our study addresses immunosuppression: a very important aspect of all therapeutic agents attempting to target aberrant inflammation. Chronic treatment with anti-inflammatory agents can lead to overall immune suppression and increased susceptibility to infections. To determine whether our therapeutic approach impacts immune responses to pathogens, we infected polycationic polymer treated animals with PR8 influenza to mimic human flu infection. We did not observe immune-suppression in treated animals, as polycationic polymers treated lupus prone mice were able to recover similarly to untreated controls. To our surprise C57B16 animals that received lethal doses of PR8 in the presence of polycationic polymer did not succumb to infection at the same rate as the control treated animals. These finding suggest that polycationic polymers may have broader application in not only controlling aberrant inflammation but in also improving the immune response to pathogens.

Thus polycationic polymers represent novel agents to potentially treat SLE as well as a wide variety of infectious diseases particularly those caused by highly pathogenic viruses such as pandemic influenza and Ebola. In the Examples we also demonstrate that administration of polycationic polymers is also capable of rescuing mice from lethal nucleic acid-induced inflammatory shock. Thus we expect that the polycationic polymers may be useful to treat viral or bacterial infections in which septic shock or large inflammatory responses are at least partially responsible for the pathology of the disease.

### Methods of Using Polycationic Polymers for Treating Inflammatory Effects of Radiation Therapy or Exposure to Radiation

We have explored the use of polycationic polymerns to limit acute inflammatory shock and disseminated intravascular coagulation that are engendered following radiation exposure, specifically after 24 hours. Recent studies have demonstrated that lethal doses of ionizing radiation cause a release of both mitochondrial (mt) and nuclear DNA into the extracellular space and circulation.

As mentioned previously, extracellular DNA and RNA from damaged cells have been increasingly implicated in pathological inflammation and activation of the coagulation system, two hallmarks of Acute Radiation Syndrome. Recent studies by our group has illustrated that cationic polymers that can scavenge such extracellular nucleic acids can limit inflammation, counteract inflammatory shock and inhibit activation of the coagulation system and limit micro- and macro-vascular thrombosis. Our data demonstrates that these molecules dramatically improve survival when administered 24 hours following total body irradiation (TBI).

As used herein, the term Acute Radiation Syndrome (ARS), also referred to as radiation toxicity or radiation sickness, refers to the acute illness caused by irradiation of part, some, most or entire body by a high dose of penetrating radiation in a very short period of time. In some cases, usually a matter of minutes is all that is required to induce radiation sickness. As used herein, the term “lethal dose of radiation” refers to the dose or radiation expected to cause death to 50% of an exposed population with 30 days (LD 50/30). Typically, the LD 50/30) is in the range of from about 400 to 450 rem (4 to 5 sieverts) that is received over a very short period of time (e.g., matter of minutes).

### Miscellaneous

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### EXAMPLES

The following examples are meant only to be illustrative and are not meant as limitations on the scope of the invention or of the appended claims.

### Example 1

### Preparation of Conjugated Polycationic Polymers

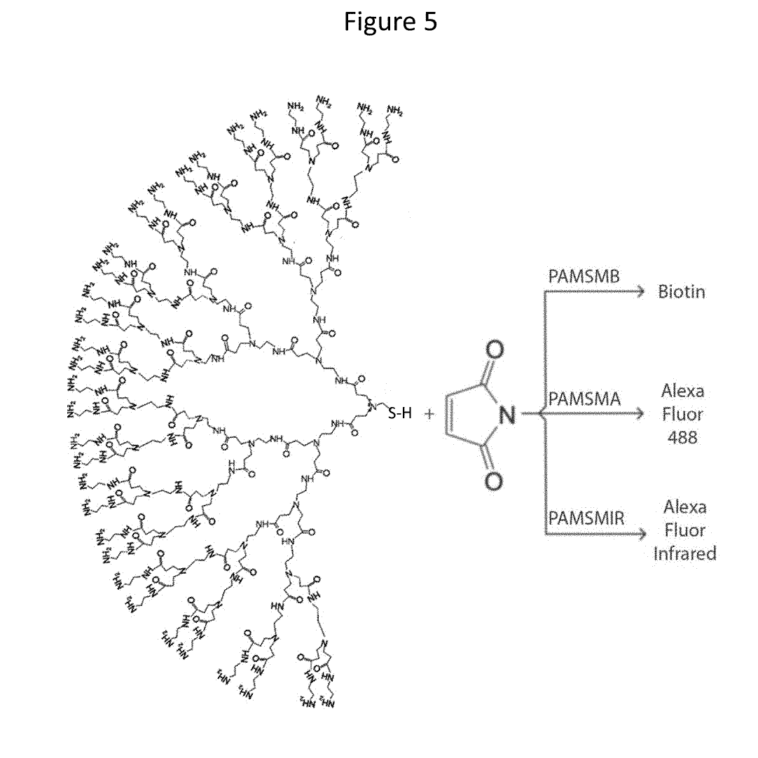

We synthesized several conjugates using a schematic similar to the generation of our biotinylated polycationic polymer (FIG. 5). Preliminary experiments indicate successful conjugations of the polycationic polymers with 1) Alexa Fluor 488 and 2) Alexa Fluor 750 Infrared which we will refer to as PAMSM-AF488 and PAMSM-AF750 respectively.

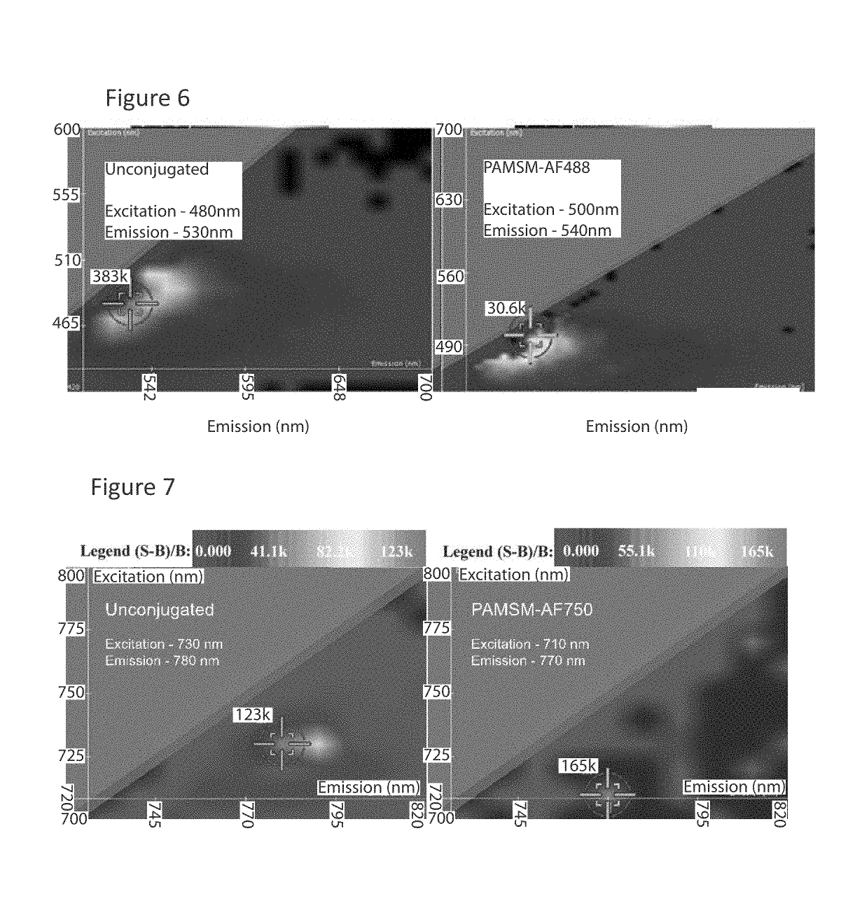

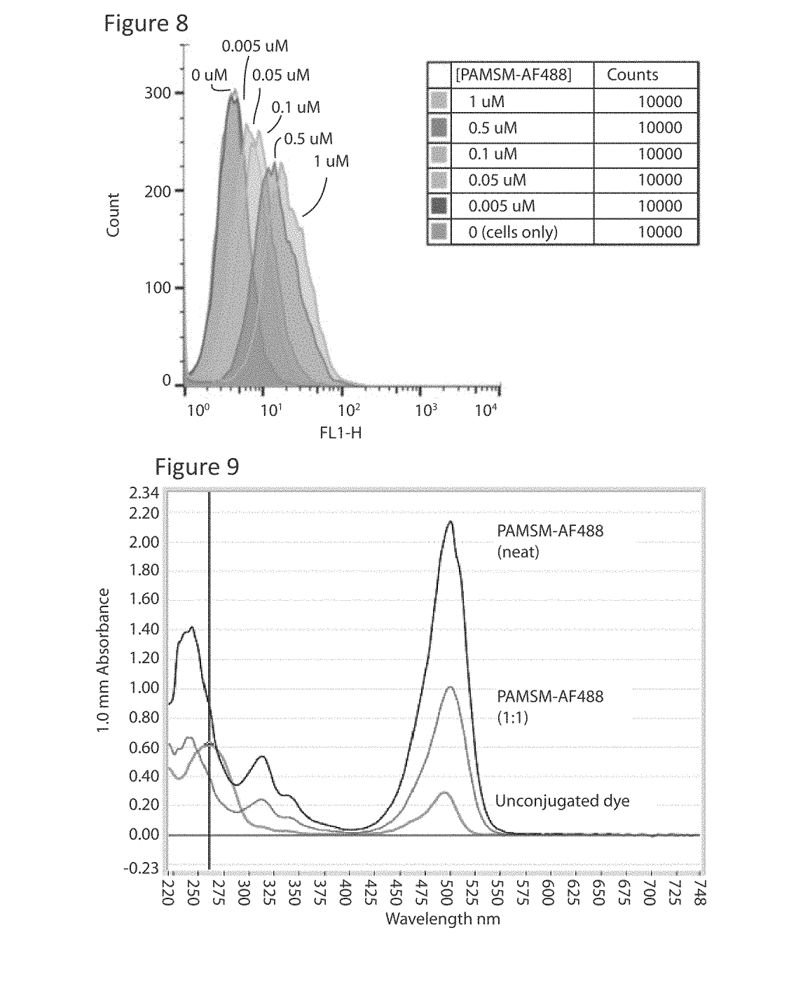

Characterization studies on the polymers demonstrate a successful conjugation event. Shifts in excitation and emissions between the unreacted dyes and polycationic polymers are indicative of covalent bonding (FIGS. 6, 7). The optimized wavelengths are displayed in spectrograph heat map. Additionally, flow cytometry was performed by incubating PAMSM-AF488 at varying concentrations with murine macrophages. A liner relationship between polymer concentration and fluorescent intensity was observed suggesting proper linking of the Alexa Fluor 488 to the polycationic polymers (FIG. 8). Relative PAMSM-AF488 concentrations were determined through Baer's law using manufacturer provided extinction coefficients and absorbance readings (FIG. 9).

Synthesis using PAMAM G4 disulfide core as a starting material began by cleaving the disulfide bond using TCEP-HCL, resulting in a hemi-PAMAM G4 thiol. The intermediate PAM-SH was then reacted with maleimide conjugates at room temperature overnight with tri-methyl amine as a catalyst. Purification was performed by dialysis using specific molecular weight cutoffs dialysis membranes for 48 hours at 4° C. Lastly, the product was frozen and lyophilized for characterization studies and preliminary experiments. All reactions were performed in water unless otherwise specified.

Optimization of excitation and emission wavelengths was performed using SpectraMax i3 plate reader and the manufacturer's optimization software where the dyes were added to a 96-well all black plate and maximum excitations and emissions were plotted as a spectrograph heat map. See FIGS. 6 and 7. Concentrations of PAMSM-AF488 were extrapolated based on the absorbance values using NanoDrop Spectrophotometer and software. Alexa Fluor 488 extinction coefficients were obtained from the manufacturer and combined with Baer's law using a 1 cm path length for the calculations. See FIG. 9. Assuming a (1:1) stoichiometric ratio between PAM-SH and maleimide dye the calculations for dye molarity was the same value used for PAMSM-AF488.

### Example 2

### Detection of Conjugated Polycationic Polymers

Flow cytometry was performed with live murine macrophage (RAW) cells that were incubated with diluting concentrations of PAMSM-AF488. Cells were seeded at 1×10⁵ cells/well in 12-well polystyrene plate overnight. Conjugated polycationic polymers were incubated with the cells for 30 min followed by extensive washing and trypsonization with PBS. Samples were then washed further before being analyzed by FACSCalibur in FL-1 (green) channel. See FIG. 8.

### Example 3

### Scavenging DAMPs with Conjugated Polycationic Polymers

Using a biotinylated cationic polymer (PAMSMB) with pancreatic cancer cells lines in vitro we are successful in scavenging pro-oncogenic damage associated molecular patterns (DAMPs) as seen by a reduction in toll-like receptors (TLRs) 3, 4, 7, and 9 activation.

Conditioned DAMP containing media is generated by exposing pancreatic cancer cell lines to radiation and allowed to incubate for three days, followed by the addition of a biotinylated cationic polymer conjugated to a streptavidin coated magnetic bead. The resultant supernatant is placed on reporter cells expressing TLR 3, 4, 7, 9 and a reduction in TLR stimulation was clearly observed in the samples that were treated with the polymer.

Inhibition of TLR activation was also seen using pancreatic cancer patient serum before and after radiation after ex-vivo scavenging. Though the difference in TLR stimulation between sera with and without polymer scavenging was not as pronounced when compared to the conditioned media samples, there was still a statistically significant difference suggesting the potential use of this biotinylated polymer as a potential therapeutic.

### Example 4

### Reversible Displacement of Negatively Charged Molecules from Conjugated Polycationic Polymers

Using the biotinylated form of the polymer we have established a protocol to displace the DAMPs that are bound to the polymer using low-molecular weight heparin (LMWH). This method was validated by electrophoretic mobility shift assay (EMSA) and TLR stimulation.

DNA in plasmid form was bound to PAMAM-G3 via co-incubation at room temperature. Binding was verified via visualization on agarose gel. LMWH displacement was executed at 60° C. with agitation of the DNA-polymer complex. Displacement of the DNA was also verified by agarose gel visualization. This same process was repeated with TLR reporter cell assays wherein the successful binding of the DNA-polymer complex resulted in low TLR activity and successful displacement with LMWH resulted in elevated TLR activation. LMWH resulted in no activation alone.

### Example 5

### PAMAM-G3 Treatment of Lupus-Prone Mice (NZBW F1) Exhibiting Chronic Skin Inflammation Significantly Ameliorates CLE-Like Phenotype

We hypothesized that polycationic polymers might represent a useful treatment strategy for systemic lupus erythematosus (SLE) since SLE is associated anti-nucleic acid autoantibodies. Therefore we evaluated the therapeutic potential of the polycationic PAMAM-G3 in NZBW F1 mice. These animals develop an autoimmune disease resembling human SLE and are characterized by high levels of circulating auto-antibodies and pro-inflammatory cytokines First, to evaluate the potential utility of a polycationic in a localized model of lupus erythematosus, we used a well-established murine tape-stripping-induced dermal injury model, which mimics cutaneous lupus erythematosus (CLE) in humans. To determine if scavengers can bind extracellular nucleic acids and thereby limit pathology following skin injury in CLE-prone mice, NZBW F1 animals were subjected to tape stripping and subsequently treated with PAMAM-G3 subcutaneously.

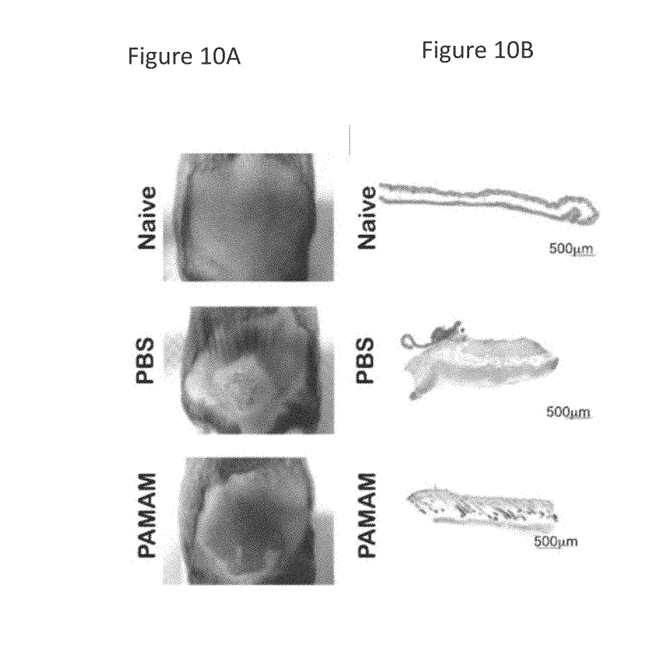

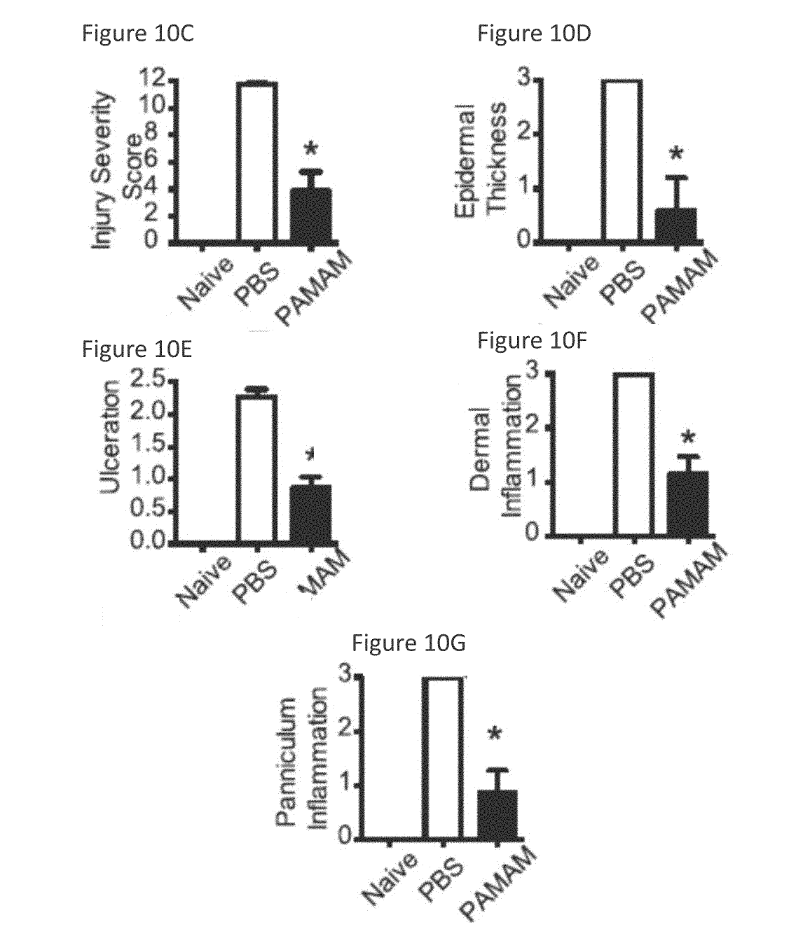

We chose to employ generation-3 PAMAM (PAMAM-G3) in these studies because it binds RNA and DNA with high affinity and blocks TLR activation by artificial nucleic acid agonists in vitro and in vivo. By contrast, lower generation PAMAM molecules, with fewer than 32 surface amine groups, were not as effective as PAMAM-G3 at inhibiting CpG DNA and poly I:C RNA-mediated activation of TLRs. Higher generation PAMAM molecules (e.g. PAMAM-G5), are as efficacious as PAMAM-G3 at inhibiting nucleic acid-mediated TLR activation but they are associated with increased toxicity making it challenging to perform studies with them in lupus prone mice. To choose a therapeutic dosing regimen, we first determined the maximum tolerated dose (MTD) of PAMAM-G3 in NZBW F1 mice (100-200 mg/kg) and then administered this polycationic polymer 5-10 fold below the MTD. The CLE-prone mice were injured and then treated with PAMAM-G3 (20 mg/kg) twice per week for 14-21 days post injury. Strikingly, subcutaneous administration of PAMAM-G3 allowed CLE-prone mice to recover from skin damage much more effectively than control animals (FIGS. 10A, 10B). Moreover, blinded histopathological analyses of skin samples demonstrated that CLE-prone mice treated with PAMAM-G3 displayed significantly lower disease grades in all pathological categories as compared to PBS treated counterparts (FIGS. 10C-10G).

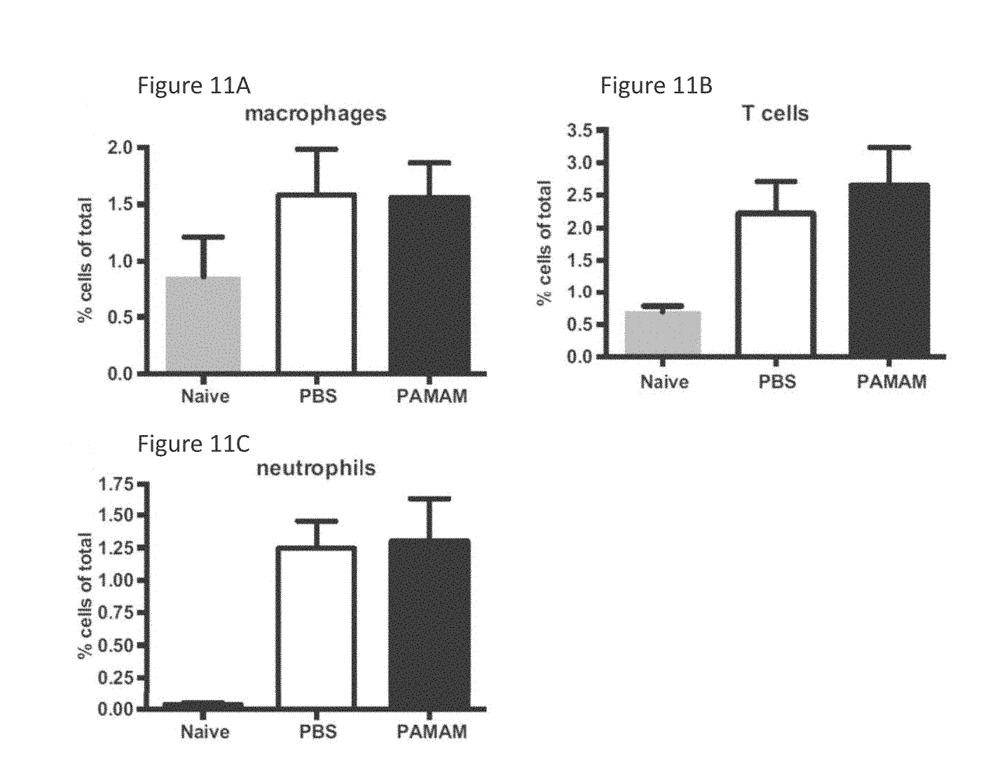

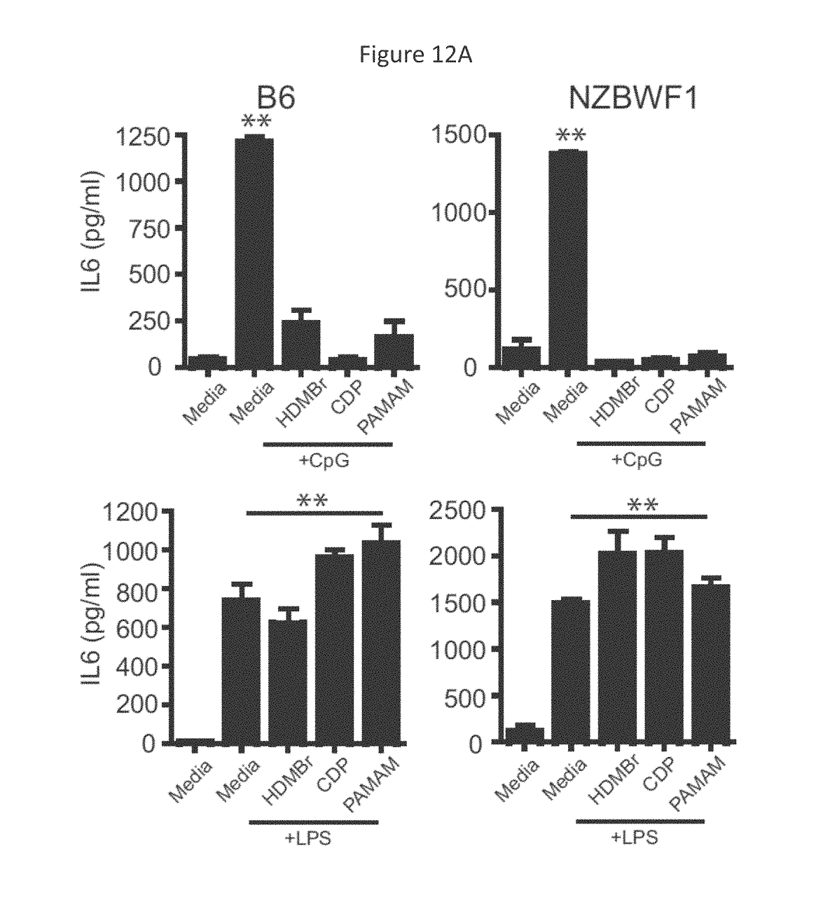

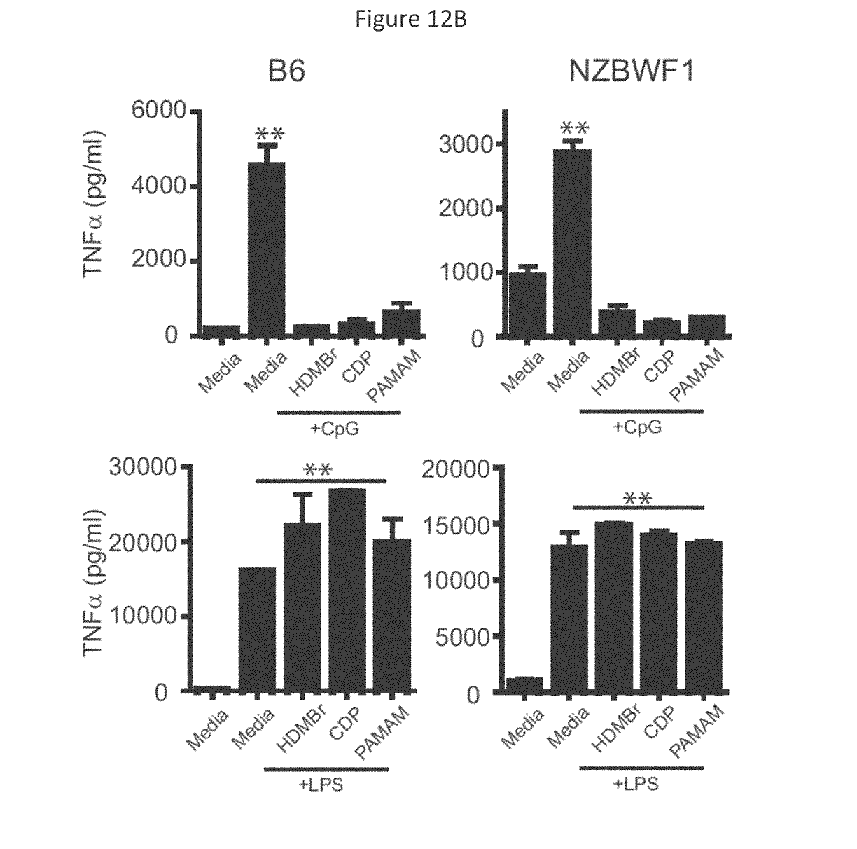

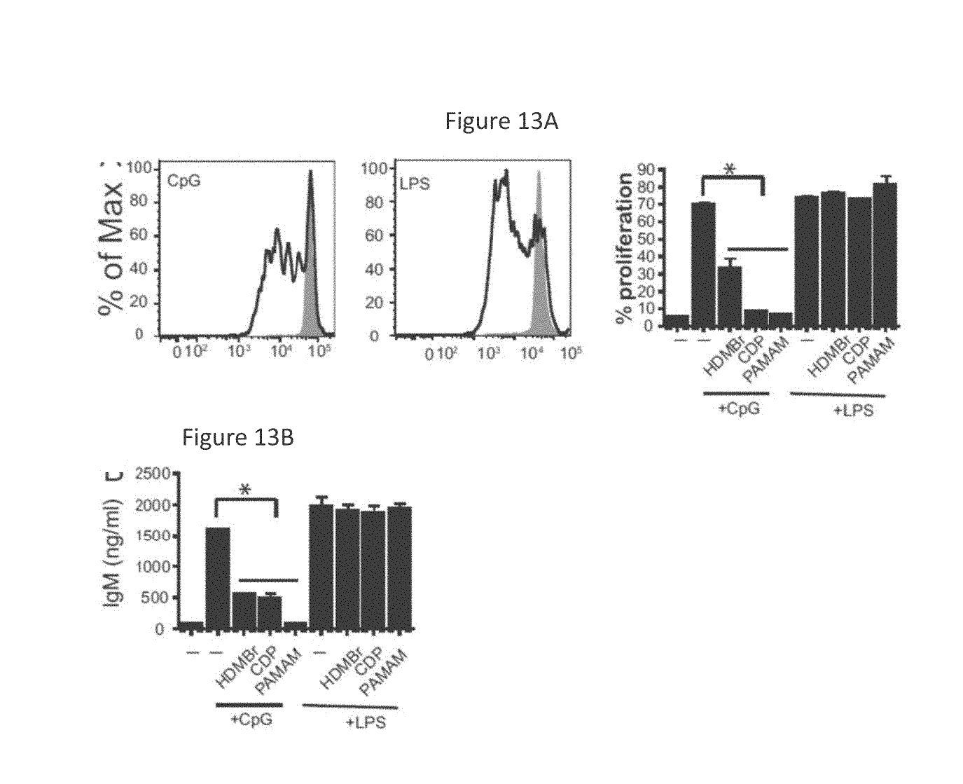

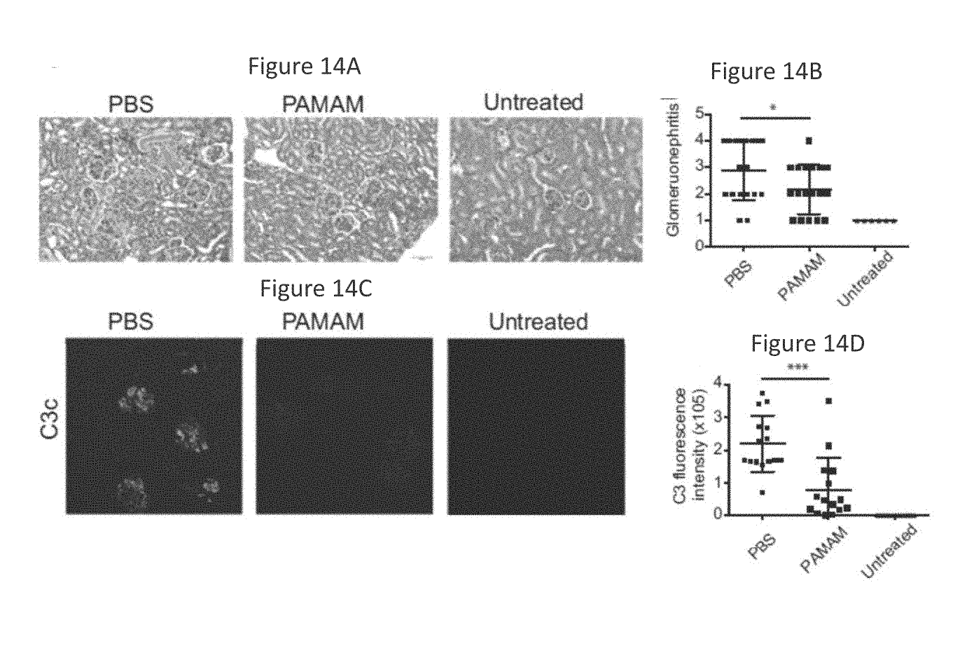

Previous studies in this CLE-prone mouse model have shown that skin damage leads to pronounced immune cell recruitment to the site of injury within 24 hours and that the resulting skin inflammation is dependent on signaling through nucleic acid sensing TLRs (TLR7 and TLR9). Therefore, we evaluated whether immune cell recruitment is affected by polycationic polymer treatment. NZBW F1 skin samples were obtained 24 hours post tape stripping and treatment with the polycationic polymer and the presence of immune cell infiltrates was analyzed. As shown in FIG. 11, PAMAM-G3 treatment does not affect immune cell (macrophages, neutrophils, dendritic cells-DCs and T cells) infiltration into damaged skin. Therefore since the inflammatory cells are present in the damaged skin, we next evaluated whether PAMAM-G3 could inhibit activation of the nucleic acid sensing TLRs on inflammatory cells taken from the NZBW F1 CLE-prone mice as compared to wild type C57/BL6 animals. As shown in FIG. 12, scavengers block TLR activation by nucleic acid agonists but not LPS in DCs isolated from NZBW F1 animals similarly to wild type animals. Thus polycationic polymer s can block nucleic acid-driven immune cell activation and inflammatory cytokine production even in a genetic background that is predisposed to rapidly respond to inflammatory triggers (FIG. 12). However, TLR-independent NZBW F1 B cell activation is unaltered by PAMAM-G3 (FIG. 13). Collectively these skin damage studies indicate that local administration of s facilitates wound healing in a CLE-prone animal following injury by scavenging extracellular nucleic acids and reducing TLR7 and TLR9 activation and this inhibition in turn limits sustained aberrant inflammation.

### Example 6

### Systemic PAMAM-G3 Treatment Reduces Glomerular Immune Complex Pathology and C3c Deposits in MRLlpr Mice

Next we sought to determine if polycationic polymers can be useful for treatment of chronic inflammatory disease in a murine model of SLE. We evaluated the ability of PAMAM-G3 to reduce glomerulonephritis and circulating auto-antibody levels, hallmarks of SLE, in MRL^{lpr} mice. As MRL^{lpr} mice spontaneously develop SLE over a few months, we started treating 10-12 week old male MRL^{lpr} mice twice a week with an intraperitoneal injection of PAMAM-G3 (20 mg/kg). After 10 weeks of polycationic polymer treatment, SLE-prone mice were analyzed for kidney damage, immune complex deposition in the kidney and levels of serum autoantibodies. As shown in FIG. 14, histological evaluation of kidney samples demonstrated that PAMAM-G3 treatment significantly reduces glomerulonephritis and kidney damage in MRL^{lpr} mice at 5 months of age (FIGS. 14A, 14B).

Since polycationic polymer treatment reduced kidney damage and glomerulonephritis, we next assessed how polycationic polymer treatment impacted complement deposition in the kidneys of MRL^{lpr} mice. By 5 months of age, immunofluorescent staining for complement (C3c) deposits revealed that all lupus-prone mice not treated with a polycationic polymer had developed C3c deposits throughout the glomerulus (FIG. 14C). By contrast, polycationic polymer treatment dramatically reduced deposition of complement C3c in the lupus-prone mice (FIGS. 14C, 14D). Collectively these data show that polycationic polymer treatment can slow SLE development and glomerulonephritis in MRCP^{lpr} animals. These results highlight the potential utility of the polycationic polymer approach to treat chronic inflammatory diseases by limiting extracellular nucleic acid debris-induced chronic activation of pro-inflammatory signaling cascades and by breaking up nucleic acid-containing immune complexes to limit their deposition in the kidney or other organs.

### Example 7

### Systemic PAMAM-G3 Treatment Decreases ANAs and Anti-dsDNA Abs in MRL^{lpr} Mice

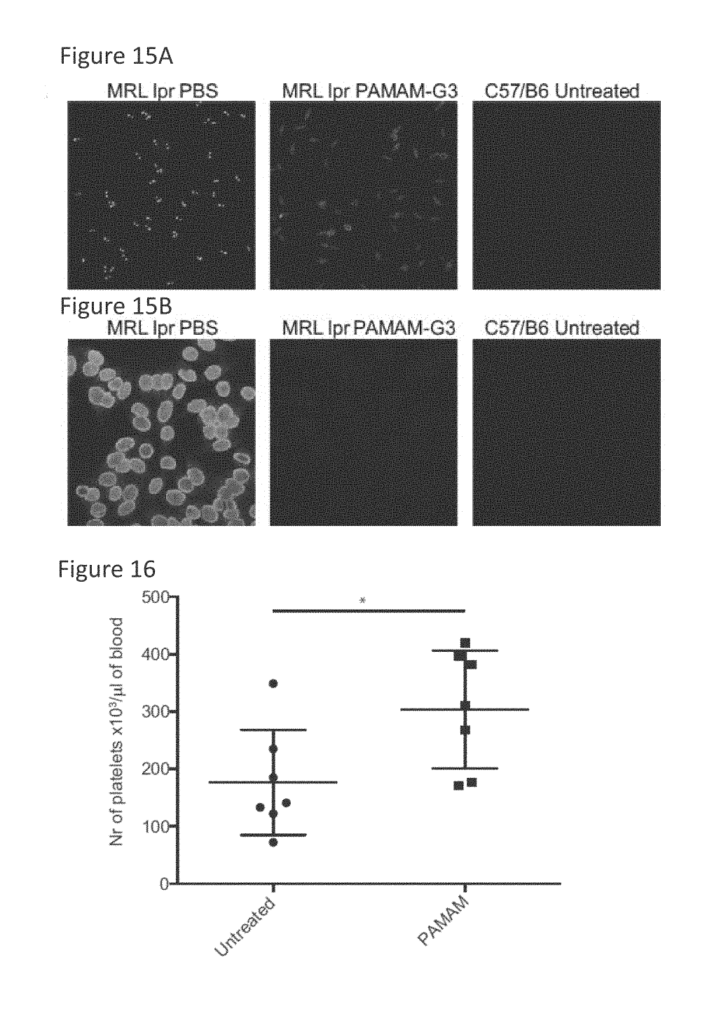

In addition to renal dysfunction, progression of SLE in the MRCP^{lpr} animals is characterized by increasing levels of circulating anti-nuclear and anti-DNA antibodies which further exacerbates autoimmunity in the lupus prone animals. To determine if long-term treatment with a polycationic polymer can impact the levels of anti-nuclear and anti-DNA antibodies in aging animals-prone to SLE, we treated MRCP^{lpr} mice with PAMAM-G3 (20 mg/kg twice a week) for 2.5 months and performed ELISA and immunofluorescent assays on serum collected from the treated animals to determine autoantibody levels. The fluorescent antinuclear antibody (ANA) assay is clinically relevant and the most sensitive approach to detect serum antibodies against a variety of endogenous nuclear components in their native antigenic form. As shown in FIG. 15A, ANA levels are significantly reduced in polycationic polymer treated lupus-prone mice as the staining intensity was significantly decreased, as compared to PBS treated. Next we evaluated the effect of polycationic polymer-treatment on anti-dsDNA antibody levels in lupus prone mice using an indirect immunofluorescence assay on Crithidia luciliae substrates, a clinically relevant assay utilized for detection of dsDNA autoantibodies in patients with SLE. We observed high levels of anti-DNA antibodies and intense staining when examining samples from PBS treated mice but much less intense or even absent staining when evaluating serum from PAMAM-G3 treated lupus-prone mice (FIG. 15B). We scored our slides based on an intensity scale (− absent, +/− low/intermediate, +/+ intense), Table 1.

TABLE 1
Anti-dsDNA and HEp-2 AB slides were scored for
intensity of staining

| | | | dsDNA Ab | | | ANA | | |
|---|---|---|---|---|---|---|---|---|
| Mouse | Treatment | N | (+) | (+/−) | (−) | (+) | (+/−) | (−) |
| MRL lpr | RAMAM-G3 | 24 | 6 | 13 | 5 | 2 | 16 | 6 |
| MRL lpr | PBS | 20 | 14 | 5 | 1 | 10 | 5 | 5 |
| C57/B6 | Untreated | 20 | 0 | 3 | 17 | 0 | 3 | 17 |

Autoimmune disorders, including SLE are associated with decreased levels of circulating platelets. We assessed circulating platelet counts in SLE animals post polycationic polymer treatment. As shown in FIG. 16, polycationic polymer treatment maintained platelet counts in the aging lupus prone mice while platelet counts significantly dropped in animals not treated with a polycationic polymer. This result indicates that when polycationic polymers limit circulating levels of nucleic acid containing debris, chronic platelet activation is limited and platelet counts remain normal.

### Example 8

### PAMAM-G3 Treatment Does Not Suppress the Immune System of NZBW F1 Animals During PR8 Influenza Infection but Protects C57B16/J Mice from Lethal PR8 Influenza Infection

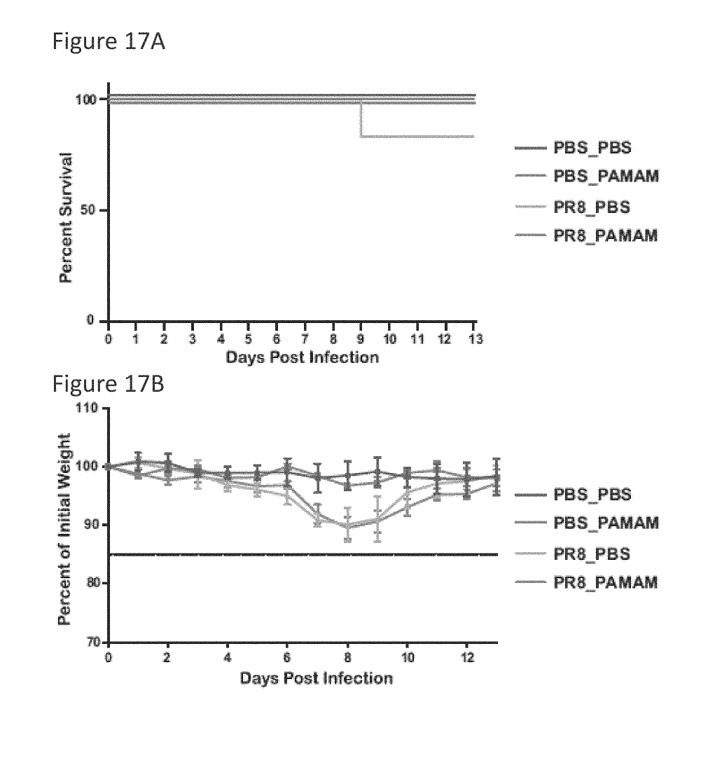

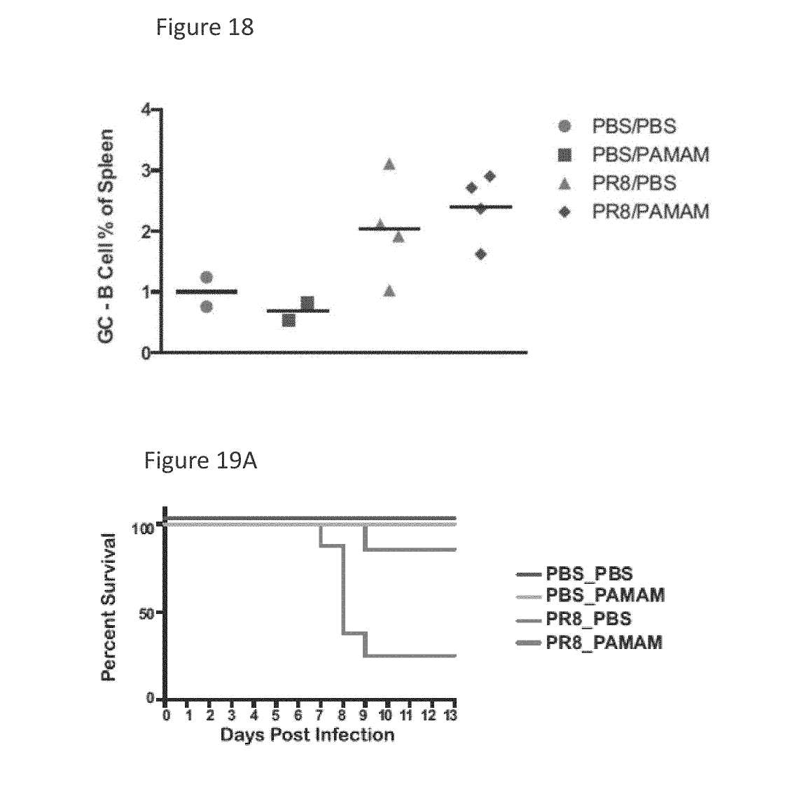

Currently marketed autoimmune disease-combatting drugs and specifically lupus treatments result in severe immune suppression and an array of side effects. To determine whether polycationic polymer treatment results in immune suppression as well, we evaluated the effects of PAMAM-G3 treatment in a viral infection model in vivo using the same dosing strategy that proved effective in the lupus-prone mice (PAMAM-G3, 20 mg/kg, 2×/week). Lupus-prone animals were challenged intranasally with the mouse-adapted influenza A H1N1 strain, PR8 at a mouse lethal dose of 10% (mLD10) to determine if polycationic polymer treatment would result in increased morbidity and mortality. Mice were monitored daily and sacrificed if they lost >15% of their body weight. Polycationic polymer treatment did not increase morbidity, as monitored by weight loss, (FIG. 17A) or mortality (FIG. 17B) following influenza infection at a mLD10. At this relatively low dose however, we observed that influenza challenge resulted in 16% fatality in lupus-prone animals not treated with the polycationic polymer while none of the polycationic polymer treated animals died. To explore the ability of these animals to mount an immune response to influenza, we further analyzed the percentages of germinal center cells in the spleen. The analysis of splenic cells indicates that germinal center maturation is also not affected by polycationic polymer treatment (FIG. 18). These data demonstrate that PAMAM-G3 treatment does not suppress the anti-viral immune response in lupus-prone animals and suggest that if adequately developed polycationic polymer s may become inherently safer, anti-inflammatory agents.

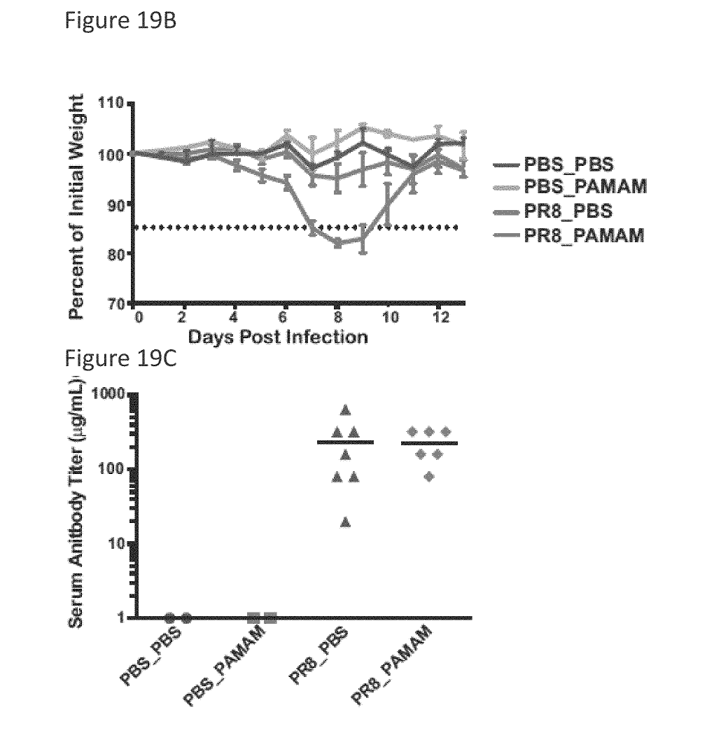

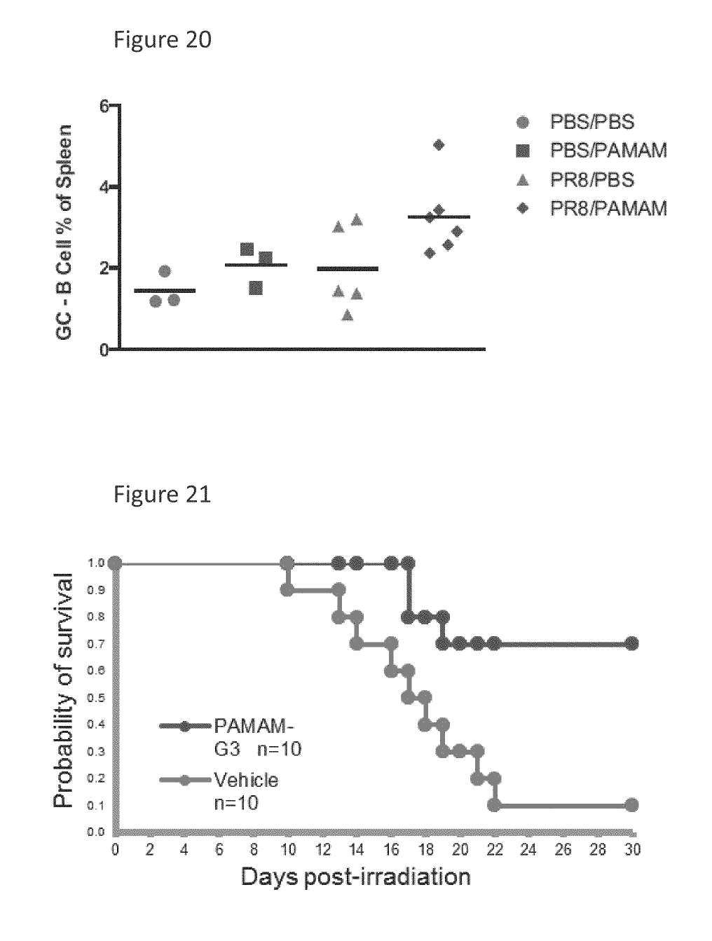

Our observation that polycationic polymer treatment may improve survival of lupus-prone mice when the animals are challenged at low mLDs, led us to investigate whether PAMAM-G3 might have beneficial effects on normal mice challenged with higher doses of influenza. Therefore, C57BL6/J mice were infected with a mLD50 of influenza A virus PR8 (H1N1) and treated with PAMAM-G3 at the time of viral challenge (20 mg/kg, 2×/week). Remarkably as shown in FIG. 19A, polycationic polymer treatment significantly improved survival following flu infection, reducing mortality from 75% to only 14% (P=0.0126). Similarly, morbidity was dramatically improved as seen by a significant reduction in weight loss following viral challenge (FIG. 19B). To explore the ability of these animals to mount an immune response to influenza, we measured the level of neutralizing antibodies to PR8 in the serum (FIG. 19C). These studies revealed that anti-influenza antibody titers are not impacted by polycationic polymer treatment. Furthermore, the analysis of splenic cells indicates that germinal center maturation is also not affected by polycationic polymer treatment (FIG. 20). These results suggest that by binding extracellular nucleic acid debris released from virally infected, dying cells, polycationic polymers can limit viral induced, acute pathological inflammation.

### Example 9

### A Scavenger Can Mitigate the Lethal Effects of Irradiation

Using the conjugated polymers provided above, we noted that the biotinylated PAMAM was able to bind to DNA both in and outside of cells. See FIG. 23A. The biotinylated polymer is stained red, the DNA is stained green and areas of overlap are yellow (see arrows). To demonstrate if polycationic polymers were able to bind inflammatory mediators released by cells after radiation exposure, we irradiated neutrophil cells with the indicated doses of radiation and then harvested conditioned media from the irradiated cells. The conditioned media was then incubated with the polycationic polymer PAMAM prior to addition to an assay for activation of TLR4. The results shown in FIG. 23B demonstrate that the addition of the polymer was able to significantly block the ability of the conditioned media to stimulate the TLR9 receptor. Similar experiments were carried out using sera taken both prior to and after radiation exposure of the subject. The patient sera was then added to cells and TLR4, TLR9, accumulation of protein in the sera or accumulation of DNA in the sera were measured. FIGS. 23C and E are similar experiments to that shown in FIG. 23B but use patient serum collected before and after radiation and easure TLR 4 and TLR 9 activation by the patient sera and demonstrate that the polymer reduces activation of the TLR receptor in both cases. FIG. 23D is a graph showing the protein levels in the patient sera and FIG. 23F is a graph showing the DNA levels present in the sera. In both cases the addition of the polymer reduces the protein and DNA levels found in the sera.

We evaluated the ability of the polycationic polymer PAMAM-G3 to rescue mice subject to lethal irradiation. BALB/c mice received 7.25 Gy of total body radiation. Mice were then treated by subcutaneous administration of the polycationic polymer PAMAM-G3 at a dose of 20 mg/kg or PBS vehicle 24 hours after irradiation. The treatment was repeated on day three after irradiation. Survival was monitored daily. Each group contained 10 animals. Similar experiments were obtained twice. This survival difference between PAMAM-G3 and the Vehicle treated groups is statistically significant with a P value=0.0057.

### Example 10

### Polycationic Polymers can Rescue Mice from Inflammatory Shock

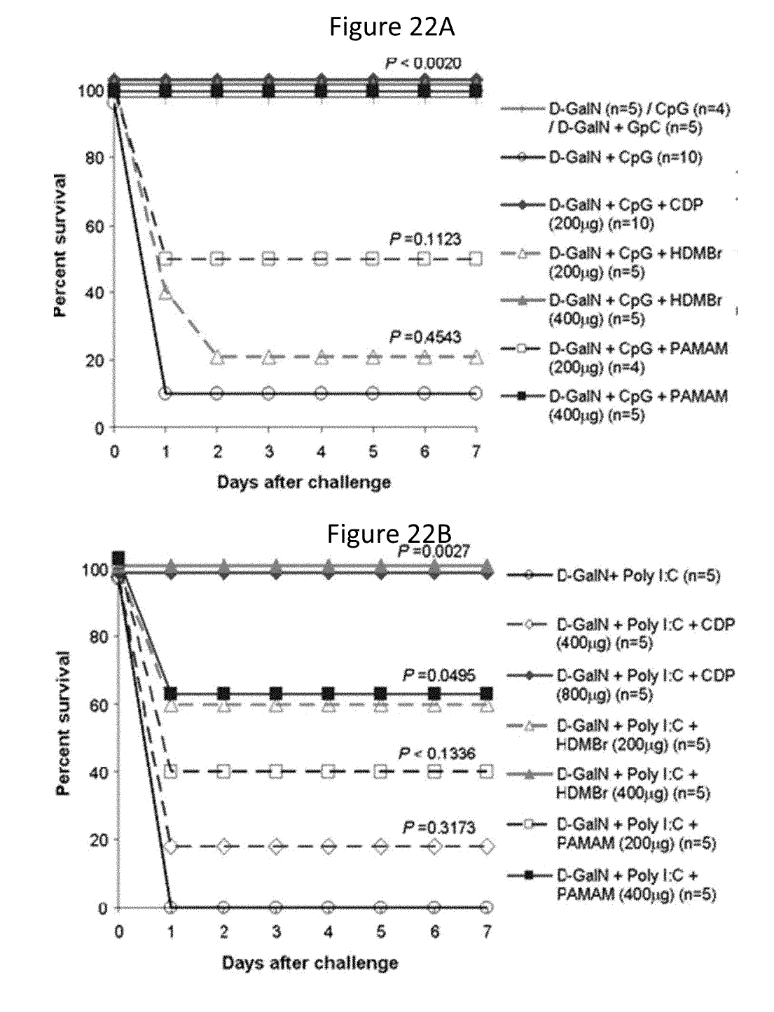

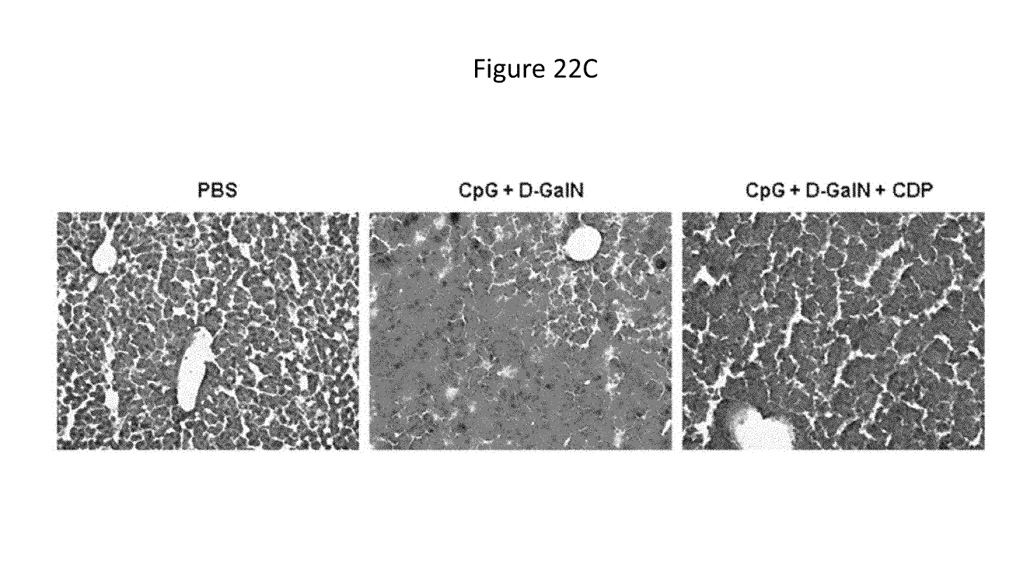

In 2009, we made the surprising observation that certain nucleic acid binding polymers can reverse nucleic acid aptamer binding to its target protein. This discovery led us to examine whether such polymers could act as NASs and inhibit the activity of proinflammatory nucleic acids that bind proteins involved in innate immunity. First, we examined whether such NASs could inhibit the activities of potent, prototypic nucleic acid molecules that are known to activate NA-sensing TLRs (TLR3, 7, 8 and 9). We discovered that certain NASs (named CDP, HDMBr, PAMAM-G3, PPA-DPA, poly L-lysine and protamine sulfate) can indeed counteract the activities of multiple nucleic acid based TLR agonists in vitro and in vivo (FIG. 22) including CpG oligos, dsRNA (Poly I:C) and ssRNA/lipid complexes (ssRNA40/LyoVec) that mediate their effects through different TLRs. Moreover as is strikingly shown in FIG. 22, several of these scavengers are able to counteract even lethal doses of proinflammatory nucleic acids in a murine acute inflammatory shock model when administered at doses as low as 8 mg/kg or 25-fold lower than shown to be safe for a single dose of the NAS CDP in mice and 3-fold lower than shown to have minimal toxicity following repeat iv administration of CDP-containing particles in non-human primates for 18 days. As shown in FIG. 22, CpG DNA and poly I:C RNA-mediated fatal liver inflammation can be counteracted by nucleic acid scavengers. FIG. 22A shows mice (n=5/group) were i.p. injected with the liver toxin D-GalN alone, CpG 1668 alone, D-GalN+control GpC oligo or D-GalN+CpG 1668. After 10 minutes, PBS (circles), NAS CDP (blue diamonds), NAS HDMBr (red triangles) or NAS PAMAM-G3 (black squares) was administered into mice challenged with D-GalN+CpG 1668 at the concentrations shown. In FIG. 22B poly I:C and D-GalN were injected i.p. into mice. NAS CDP (blue), NAS HDMBr (red) or NAS PAMAM-G3 (black) was injected and mice were monitored daily for survival. In FIG. 22C liver pathology of mice injected with PBS, CpG 1668+D-GalN or CpG 1668+D-GalN+NAS CDP. Sixteen hours following injection, liver specimens were collected for histological studies (hematoxylin and eosin staining). Magnification ×20.

### Materials and Methods for Examples 5-8

### Mice

C57BL/6, NZBW F1 and MRLlpr were obtained from the Jackson Laboratory (Bar Harbor, Me.). Mice were housed in a pathogen-free barrier facility at Duke University. Only male mice were used in all of our studies.

### SLE and Flu Studies

Tape Stripping: Tape stripping was performed on the dorsal area of the mice, post shaving, with standard size bandages, 20 strokes. PAMAM-G3 (20 mg/kg) (Sigma-Aldrich) was administered at the time of tape-stripping subcutaneously and every three days after injury. Animals were sacrificed at 14 days following injury. All skin samples were then harvested and fixed in 10% formalin for future histological analysis.

SLE long-term treatment: MRL-lpr male mice were injected with PAMAM-G3 (20 mg/kg) twice a week for a period of 8-10 weeks starting at 10 weeks of age. Mice were then sacrificed and blood and tissue were collected for further analysis.

PR8 infections: Mouse-adapted virus strain, influenza A/Puerto Rico/8/34 (H1N1; PR8) was obtained from Charles River. 10-week mice were anesthetized with vaporized isoflourane. Virus was administered intra-nasally in a total volume of 40 μL sterile pharmaceutical grade saline. Control mice were mock treated with pharmaceutical grade saline only. PAMAM-G3 was injected intraperitoneally at 20 mg/kg in a total volume to 200 μL of pharmaceutical grade saline. Weight loss and survival of infected mice was followed over a period of 14 days. Mice that lost 15% or more of their body weight were euthanized and recorded as dead per Duke University Institutional Animal Care and Use Committee guidelines.

### Cell Culture

B cell activation: B cells from spleens for NZBW F1 animals were purified by negative selection. Stimulation assays were performed as previously described (20)

DC culture: Murine bone marrow DCs were isolated from NZBW F1 mice and were cultured in the presence of GM-CSF (Peprotech) and IL-4 (Peprotech) as previously described (55). Stimulation assays were performed as previously described (20).

### Neutralization Assay

Virus microneutralization assay was performed as described previously (56) with modifications. Briefly virus and serum dilutions were performed as described and then mixed with 100 μl of freshly trypsinized MDCK-London cells containing 1.5×104 cells in 96-well cell culture treated plates. Negative controls consisted of cells alone, while positive controls contained virally infected cells. Plates were incubated for 20 hours before fixation with acetone. Endogenous biotin in wells was blocked with PBS containing 0.1% avidin (Life Technologies) for 15 minutes followed by washes and any bound avidin was blocked PBS containing 0.01% biotin (Sigma Aldrich) for 15 minutes. Plates were analyzed for positive infection via ELISA. Mouse monoclonal biotinylated anti-NP antibodies MAB8257B and MAB8258B (Millipore), dilution 1:6,000, and HRP-streptavidin conjugate (BD Biosciences), dilution 1:4,000 were used in the ELISA. Color was developed using OPD substrate (Sigma-Aldrich) in citrate buffer (Sigma-Aldrich), and optical density was measured at 490 nm in plate reader (Molecular Devices,). The highest serum dilution that generated >50% specific signal was determined to be the neutralization titer. 50% specific signal=(OD490 virus control—OD490 cell control)/2+OD490 cell control.

### Determination of Clinical Disease

Skin lesion scoring was conducted in a blinded fashion by a trained veterinarian pathologist as previously described (28, 29). Briefly, skin samples were collected, fixed in 10% formalin, paraffin embedded (FFPE) and further processed for H+E staining. Epidermal thickness, degree of ulceration, intraepithelial inflammation, dermal inflammation and panniculus inflammation were assessed and graded on a scale from 0 to 3:0-normal skin architecture, 1-mild inflammation with slight epidermal hyperplasia, 2-moderate inflammation with noticeable epidermal hyperplasia-3-severe inflammation with marked epidermal hyperplasia. All parameters were scored separately and summed to reach a total disease score.

For kidney disease, kidneys were collected and further processed as FFPE samples for H+E staining or snap frozen in OCT for immunofluorescence staining. Glomerulonephritis scoring was done as previously described in a blinded fashion by a trained veterinarian pathologist (7). Briefly, kidneys were scored for glomerulonephritis on a scale of 1 to 4: 1-normal, 2-mild, 3-moderate and 4-severe. This scoring takes into account glomerular changes, interstitial changes and severity of lymphoplasmatic infiltration into the kidney.

### Immunofluorescence

Slides coated with Crithidia luciliae (Scimedx) were rehydrated with PBS for 30 minutes. Samples were then blocked for two hours (PBS, 0.1% Tween-20, 5% goat serum and 1% rat anti-mouse CD16/CD32). Serum samples were subsequently added to the slides at various dilutions (1:40-1:360). Serum Ab levels were detected using secondary antibody goat anti-mouse IgG FITC. Kidneys from treated animals were processed as described above. 5 μm sections were then stained with anti-complement antibody. All slides were mounted and images were acquired using a Zeiss Axiovert 500 confocal immunofluorescent microscope. Images were analyzed for staining intensity using image J.

### Antibodies and FACS

Monoclonal Abs included: B220, CD45.2, CD4, CD8, IFN□, CD3, CD11c and GL7 (eBioscience). Single cell suspensions of cultured cells were counted and 106 cells were suspended in FACS buffer (PBS plus 2% FBS) and stained with various antibody combinations. In addition skin samples collected at 24-post injury were enzymatically digested with 0.28 u/ml Liberase 3 (Roche) for 30 minutes at 37° C. then treated with DNAse, filtered and stained with various antibodies. Flow cytometry was performed on a Gallios flow cytometer and FACSCanto. All data was analyzed with FlowJo software.

### Statistical Analysis

Statistical significance was determined with two-tailed Student's t test or analysis of variance (ANOVA). Long-rank Mantel-Cox test was performed on all survival curve graphs. All p values less than 0.05 were considered significant.

### REFERENCES

1. Jaewoo L, Jang Wook S, Ying Z, Kam W L, David P, and Bruce A S. Nucleic acid-binding polymers as anti-inflammatory agents. Proceedings of the National Academy of Sciences. 2011; 108(34): 14055-60.

2. Leon S A, Ehrlich G E, Shapiro B, and Labbate V A. Free DNA in the Serum of Rheumatoid arthritis Patients. J Rherumatol. 1977; 4: 139-143.

3. Vaz J, and Andersson R. Intervention on Toll-Like Receptors in Pancreatic Cancer. World Journal of Gastroenterology. 2014; 20 (19) 5808-5817.

4. Holl E K, Bond J E, Selim M A, Ehanire T, Sullenger B, and Levinson H. The Nucleic Acid Scavenger Polyamidoamine Third-Generation Dendrimer Inhibits Fibroblast Activation and Granulation Tissue Contraction. Plastic and Reconstructive Surgery. 2014; 134(3):420-33.

5. Holl E K, Shumansky K L, Borst L B, Burnette A D, Sample C J, Ramsburg E A, and Sullenger B A. Scavenging Nucleic Acid Debris to Combat Autoimmunity and Infectious Disease. Proceedings of the National Academy of Sciences. 2016.

6. Gautschi O, Bigosch C, Huegli B, Jermann M, Marx A, Chasse E, Ratschiller D, Weder W, Joerger M, Betticher D C, Stahel R A, and Ziegler A. Circulating deoxyribonucleic acid as prognostic marker in non-small-cell lung cancer patients undergoing chemotherapy. J Clin Oncol. 2004; 22:4157-4164.

7. U.S. Pat. No. 9,340,591 to Sullenger et al. (issued 17 May 2016).

8. U.S. patent application Ser. No. 13/496,313 to Sullenger et al. (published as 2012/0183564 19 Jul. 2012).

9. International Application Ser. No. PCT/US2014/033509 to Sullenger et al. (published as WO/2014/169043 16 Oct. 2014).

10. International Application Ser. No. PCT/US2015/026201 to Sullenger et al. (published as WO/2015/161094 22 Oct. 2015).

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A conjugated polycationic polymer, the conjugated polycationic polymer comprising:
(a) a dendron, the dendron comprising a focal point, a plurality of cationic termini, and a branched cationic polymer between the focal point and the plurality of cationic termini;
(b) a detectable label; and
(c) a crosslinker, wherein the crosslinker links the detectable label and the focal point of the dendron.

2. The conjugated polycationic polymer of claim 1,
wherein the dendron further comprises a first crosslinkable moiety,
wherein the detectable label comprises a second crosslinkable moiety, the second crosslinkable moiety capable of crosslinking with the first crosslinkable moiety; and
wherein the crosslinker is prepared by contacting the first crosslinkable moiety with the second crosslinkable moiety.

3. (canceled)

4. (canceled)

5. The conjugated polycationic polymer of claim 1, wherein the cationic polymer is selected from the group consisting of a poly(β amino ester), disulfide containing poly(β amido amine) or poly(β hydroxyl amine).

6. (canceled)

7. (canceled)

8. (canceled)

9. (canceled)

10. (canceled)

11. The conjugated polycationic polymer of claim 1, wherein the detectable label is a binding label, a chromophore, an enzyme label, a bioluminescent label, a quencher, or a radiolabel.

12. (canceled)

13. (canceled)

14. (canceled)

15. A scavenging apparatus comprising:
(a) a plurality of conjugated polycationic polymers and
(b) a substrate, wherein the plurality of conjugated polycationic polymers are immobilized on the substrate.

16. The scavenging apparatus of claim 15, wherein the plurality of conjugated polycationic polymers are the polycationic polymers of claim 1.

17. The scavenging apparatus of claim 16,
wherein the substrate comprises a binding moiety; and
wherein the detectable label binds with the binding moiety to immobilize the polycationic polymer on the substrate.

18. (canceled)

19. (canceled)

20. (canceled)

21. (canceled)

22. The scavenging apparatus of claim 15, wherein the plurality of polycationic polymers comprise:
(a) a dendron, the dendron comprising a focal point, a plurality of cationic termini, and a branched cationic polymer between the focal point and the plurality of cationic termini; and
(b) a crosslinker, wherein the crosslinker links the substrate and the focal point of the dendron.

23. The scavenging apparatus of claim 22,
wherein the dendron further comprises a first crosslinkable moiety,
wherein the substrate comprises a second crosslinkable moiety, the second crosslinkable moiety capable of crosslinking with the first crosslinkable moiety; and
wherein the crosslinker is prepared by contacting the first crosslinkable moiety with the second crosslinkable moiety.

24. (canceled)

25. The scavenging apparatus of claim 15, wherein the substrate is a glass, silicon, a silicone polymer, a metal, a plastic, a magnetic substrate, or an electrospun fiber.

26. (canceled)

27. (canceled)

28. (canceled)

29. (canceled)

30. (canceled)

31. A method of using the scavenging apparatus of claim 15 comprising contacting a solution with the scavenging apparatus.

32. (canceled)

33. (canceled)

34. A method for the reduction of inflammatory mediators in a bodily fluid of a subject comprising:
contacting the bodily fluid with a polycationic polymer or scavenging apparatus,
wherein the contacting step reduces the concentration of the inflammatory mediators in the bodily fluid.

35. The method of claim 34, wherein the polycationic polymer is the conjugated polycationic polymer of claim 1 or is selected from the group consisting of PPA-DPA, CDP, CDP-Im, PAMAM, and HDMBr.

36. The method of claim 34, wherein the scavenging apparatus is the scavenging apparatus of claim 15.

37. The method of claim 34, wherein the contacting step is performed within the subject.

38. The method of claim 34, wherein the contacting step is performed outside the subject.

39. (canceled)

40. (canceled)

41. The method of claim 34, wherein the bodily fluid is blood, lymph, plasma, serum, urine or cerebral spinal fluid.

42. (canceled)

43. The method of claim 34, wherein the subject suffers from a condition selected from the group consisting of an autoimmune disease, an infectious disease, or an inflammatory effect of radiation therapy and exposure to radiation.

44. The method of claim 43, wherein the condition is selected from the group consisting of lupus, influenza and acute radiation syndrome.

45. (canceled)

46. (canceled)

47. (canceled)

48. The method of claim 34 further comprising administering a therapeutic agent to the subject.

49. The method of claim 48, wherein the therapeutic agent is selected from the group consisting of a lupus therapeutic agent, an influenza therapeutic agent and a radiation therapeutic agent.

50. (canceled)

51. (canceled)

52. (canceled)

53. (canceled)

54. (canceled)

55. A method for the tracking of a conjugated polycationic polymer, the method comprising:
contacting the conjugated polycationic polymer_with a cell in vitro or ex vivo or administering the conjugated polycationic polymer to a subject and
determining the position of the conjugated polycationic polymer relative to a cell membrane or an organelle membrane of the cell or within the subject,
wherein the conjugated polycationic polymer is the conjugated polycationic polymer of claim 1.

56.-67. (canceled)
